(19) United States
(12) Patent Application Publication
(10) Pub. No.: US 2023/0287465 A1
(43) Pub. Date: Sep. 14, 2023

(54) BIOCHEMICAL SATURATION OF MOLECULES AND ITS USE

(71) Applicant: GEAENZYMES CO., Sunnyvale, CA (US)

(72) Inventors: Javier Caceres-Delpiano, Santiago (CL); Simon Correa, Santiago (CL); Fabian Henriquez, Santiago (CL); Pedro Retamal, Santiago (CL); Juan Cristobal Jimenez, Santiago (CL); Carolina Mendez-Galvez, Santiago (CL); Cynthia Sanhueza, Santiago (CL); Leonardo Alvarez, Santiago (CL)

(21) Appl. No.: 18/299,358

(22) Filed: Apr. 12, 2023

Related U.S. Application Data
(63) Continuation of application No. PCT/US2021/071870, filed on Oct. 14, 2021.
(60) Provisional application No. 63/092,712, filed on Oct. 16, 2020.

(51) Int. Cl. C12P 7/6472; C12N 9/02

(52) U.S. Cl. CPC C12P 7/6472; C12N 9/0067

(57) ABSTRACT

Provided herein are methods and compositions for selective enzyme-based hydrogenation of molecules as an alternative for current chemical catalyst-based methods. These methods include different enzymes and their related processes followed to obtain fully saturated or partially saturated molecules, without producing unwanted stereoisomers, for example trans-fatty acids.

### CROSS-REFERENCE TO RELATED APPLICATIONS

This application is a continuation application of PCT International Application Number PCT/US2021/071870, filed Oct. 14, 2021, designating the United States of America and published in the English language, which is an International Application of and claims the benefit of priority to U.S. Provisional Application No. 63/092,712, filed Oct. 16, 2020, the disclosures of which are hereby expressly incorporated by reference in their entireties.

### REFERENCE TO SEQUENCE LISTING

The present application is being filed along with a sequence listing in electronic format. The sequence listing is provided as a file entitled GEAE007C1, created Apr. 10, 2023, which is 81 KB in size. The information in the electronic format of the sequence listing is incorporated herein by reference in its entirety.

### BACKGROUND

The present disclosure relates to the field of bioengineering and biomanufacturing. More precisely, it relates to enzyme compositions and methods of using the compositions for the production of partial or full hydrogen saturated products of interest for food, cosmetic, or pharmaceutical use, among others.

Catalytic hydrogenation reactions have become key processes in varied manufacturing industries, including agricultural, food, pharmaceutical, and fine chemicals, and the continuous innovations in hydrogenation processes highlight the importance of this technology (Machado, Curr Opinion Drug Disc Dev, 4(6), 745-755, 2001). Part of the continuous challenges in catalytic hydrogenations is the ability to maximize the activity while also maintaining high selectivity and reducing isomerization of the obtained products (Machado, (2001) Curr Opinion Drug Disc Dev, 4(6), 745-755; Patterson, (2011) Hydrogenation of Fats and Oils, pp. 33-48, AOCS Press). This has become extremely important for applications in the food industry, in which unwanted isomers of lipid hydrogenation can have important health risks if consumed (Gebauer, (2013) Encyclopedia of Human Nutrition, pp. 288-292, 3rd ed. Academic Press).

Lipids are essential biological macromolecules, the main constituents of fats and oils, and compounds of great commercial value, either as commodities or as raw materials (Baumann, (1988) Angewandte Chemie International Edition, 27(1), 41-62). The melting point of a lipid is the temperature at which it transitions from solid to liquid state. Lipids with low melting points are commonly liquid at moderate ambient temperatures (around 25° C.). On the contrary, lipids with high melting points are commonly semi-solid or solid at ambient temperature. This distinction makes different lipids appropriate for different manufacturing uses. The melting point of lipids depends on a large degree on the type of chemical bonding that occurs within the lipid hydrocarbon chain. Lipids that only contain single bonds between their carbon atoms have high melting points and are commonly referred to as saturated because single bonded carbons are saturated with hydrogen atoms (Patterson, (2011) Hydrogenation of fats and oils, pp. 1-32, AOCS Press). Other lipids can contain one or more double bonds within the hydrocarbon chain and are thus unsaturated, and have low melting points. An important market need exists for lipid products that melt between 30° to 40° C. because products with these qualities are solid at room temperature but liquid when in contact with the skin or mouth. This proves as a key quality that impacts the texture of products, a critical property for products of the food and cosmetic industries, and a property that is highly associated with product quality by consumers (Arellano, (2015) Specialty oils and fats in food and nutrition, pp. 241-270, Woodhead Publishing).

Since the beginning of the 1900s, several chemical methods have been devised that allow the chemical hydrogenation of vegetable oils, rich in unsaturated fatty acids, to generate saturated hardened fats. These processes are based on the reaction between molecular hydrogen and oils in the presence of a metallic catalyst (like palladium or nickel), and have been used to produce large yields of saturated lipids (Arellano, (2015) Specialty oils and fats in food and nutrition, pp. 241-270, Woodhead Publishing). Moreover, the same method can be applied to hydrogenate non-lipid hydrocarbon molecules, which generates products of interest for a variety of chemical industries (Baumann, (1988) Angewandte Chemie International Edition, 27(1), 41-62). One important drawback of chemical saturation of fatty acids for the food and cosmetic industry uses, is that when the process is incomplete, it can yield high amounts of monounsaturated fatty acids with a trans configuration, a geometric configuration of the carbon double bond that occurs in a low proportion in nature in comparison with the natural occurring cis configuration. Trans fats side products have different chemical and physical properties than their natural counterparts due to their chemical bond geometry, and their consumption has been linked with cardiovascular disease as well as other significant pathologies, leading several countries to impose legal trans-fat limits (Gebauer, (2013) Encyclopedia of Human Nutrition, pp. 288-292, 3rd ed. Academic Press).

As a consequence of the reduction in demand for chemically hardened vegetable oils, there has been an increase in the demand from the industry for high melting point vegetable oils and butters, including cocoa butter, shea butter and palm oil, which have the desirable hardness at room temperature and melting properties to replace chemically hardened oils. However, these alternatives are either expensive, not sustainable, and face scrutiny due to their sourcing or environmental concerns due to promotion of deforestation (Meijaard, (2020) Nature plants, 6(12), pp. 1418-1426; Clough, (2009) Conservation letters, 2(5), 197-205; Elias, (2013). Sociologia Ruralis, 53(2), 158-179). Due at least to these considerations, there is a dire need for alternative technologies that allow the controlled modification of the hydrogenation state of lipids, to generate the saturation of fatty acids without the occurrence of trans fatty acids, and to obtain greener and safer methods to manipulate lipids and their melting temperatures.

### SUMMARY

Some embodiments provided herein relate to methods that allow the hydrogenation of target molecules by means of an enzyme catalyst, through a novel repertoire of enzymes that allow modification of substrates in order to obtain products that currently can only be obtained through chemical modification, or to obtain novel products not currently available in the market. Moreover, some embodiments provided herein relate to products and methods of making these products, which have specific spatial conformations while avoiding unwanted side products (stereoselectivity), in comparison to other available chemical hydrogenation methods that generate products with mixed spatial conformation products (mixes of left- and right-handed enantiomers).

Accordingly, provided herein are methods of saturating an unsaturated molecule. In some embodiments, the methods include contacting an unsaturated molecule with an enzyme to produce a saturated molecule, and recovering the saturated molecule.

In some embodiments, the saturated molecule is fully or partially saturated. In some embodiments, the unsaturated molecule comprises an unsaturated alkene. In some embodiments, the unsaturated molecule is an unsaturated triglyceride or a free fatty acid. In some embodiments, the unsaturated molecule is vegetable oil. In some embodiments, the unsaturated molecule is olive oil or canola oil.

In some embodiments, contacting the unsaturated molecule with the enzyme is performed in a solvent. In some embodiments, contacting is performed for a sufficient period of time to allow at least partial saturation.

In some embodiments, the enzyme is in solution. In some embodiments, the enzyme is immobilized. In some embodiments, the enzyme is immobilized on a polymeric support. In some embodiments, the polymeric support is an insoluble polymer microbead.

In some embodiments, the enzyme is prepared by protein fermentation or chemical synthesis. In some embodiments, the enzyme is a purified enzyme. In some embodiments, the enzyme is a recombinant nickel binding enzyme. In some embodiments, the enzyme has an amino acid sequence as set forth in SEQ ID NOs: 1-52, or having a sequence identity of at least 75% to any one of SEQ ID NOs: 1-52. In some embodiments, the enzyme has an amino acid sequence as set forth in SEQ ID NO: 15 or 40, or having a sequence identity of at least 75% to any one of SEQ ID NOs: 15 or 40. In some embodiments, the enzyme has a sequence having 75%, 80%, 85%, 90%, 91%, 92%, 93%, 94%, 95%, 96%, 97%, 98%, or 99% sequence homology to any one of SEQ ID NOs: 1-52. In some embodiments, the enzyme includes a consensus sequence as set forth in SEQ ID NO: 53.

In some embodiments, the enzyme is a novel designed protein having hydrogenase activity and comprising a substrate specific binding site. In some embodiments, the substrate specific binding site comprises one or more alkene unsaturation sites. In some embodiments, the enzyme is a hydrogenase enzyme engineered to bind a non-canonical substrate. In some embodiments, the non-canonical substrate comprises one or more alkene unsaturation sites. In some embodiments, the hydrogenase enzyme comprises a modified hydrophobic portion that supports the recognition of an unsaturated acyl chain. In some embodiments, the enzyme is a dehydrogenase enzyme engineered to bind a non-canonical substrate. In some embodiments, the non-canonical substrate comprises one or more alkene unsaturation sites. In some embodiments, the enzyme is a desaturase enzyme. In some embodiments, the desaturase enzyme is engineered. In some embodiments, the desaturase enzyme is engineered to bind a transition metal in its active site. In some embodiments, the active site comprises at least 2 cysteine residues that support transition metal binding. In some embodiments, the transition metal is nickel, iron, or palladium. In some embodiments, the active site comprises an arginine residue that is configured to support a frustrated Lewis pair reaction.

Some embodiments relate to the following enumerated alternatives:
1. A method of saturating an unsaturated molecule, the method comprising: contacting an unsaturated molecule with an enzyme to produce a saturated molecule; and recovering the saturated molecule.
2. The method of alternative 1, wherein the saturated molecule is fully or partially saturated.
3. The method of any one of alternatives 1-2, wherein the unsaturated molecule comprises an unsaturated alkene.
4. The method of any one of alternatives 1-3, wherein the unsaturated molecule is an unsaturated triglyceride or a free fatty acid.
5. The method of any one of alternatives 1-4, wherein the unsaturated molecule is vegetable oil.
6. The method of any one of alternatives 1-5, wherein the unsaturated molecule is olive oil or canola oil.
7. The method of any one of alternatives 1-6, wherein contacting the unsaturated molecule with the enzyme is performed in a solvent.
8. The method of any one of alternatives 1-7, wherein the contacting is performed for a sufficient period of time to allow at least partial saturation.
9. The method of any one of alternatives 1-8, wherein the enzyme is in solution, or wherein the enzyme is immobilized.
10. The method of alternative 9, wherein the enzyme is immobilized on a polymeric support.
11. The method of alternative 10, wherein the polymeric support is a polymer microbead.
12. The method of any one of alternatives 1-11, wherein the enzyme is prepared by protein fermentation or chemical synthesis.
13. The method of any one of alternatives 1-12, wherein the enzyme is a purified enzyme.
14. The method of any one of alternatives 1-13, wherein the enzyme is a recombinant nickel binding enzyme.
15. The method of any one of alternatives 1-14, wherein the enzyme comprises a consensus sequence as set forth in SEQ ID NO: 53.
16. The method of any one of alternatives 1-15, wherein the enzyme has an amino acid sequence as set forth in SEQ ID NOs: 1-52, or having a sequence identity of at least 75% to any one of SEQ ID NOs: 1-52, such as 75%, 80%, 85%, 90%, 91%, 92%, 93%, 94%, 95%, 96%, 97%, 98%, or 99% sequence identity.
17. The method of any one of alternatives 1-16, wherein the enzyme has an amino acid sequence as set forth in SEQ ID NO: 15 or 40, or having a sequence identity of at least 75% to any one of SEQ ID NOs: 15 or 40, such as 75%, 80%, 85%, 90%, 91%, 92%, 93%, 94%, 95%, 96%, 97%, 98%, or 99% sequence identity.
18. The method of any one of alternatives 1-17, wherein the enzyme is a novel designed protein having hydrogenase activity and comprising a substrate specific binding site.
19. The method of alternative 18, wherein said substrate specific binding site comprises one or more alkene unsaturation sites.
20. The method of any one of alternatives 1-19, wherein said enzyme is a hydrogenase enzyme engineered to bind a non-canonical substrate.
21. The method of alternative 20, wherein said non-canonical substrate comprises one or more alkene unsaturation sites.
22. The method of any one of alternatives 20-21, wherein the hydrogenase enzyme comprises a modified hydrophobic portion that supports the recognition of an unsaturated acyl chain.
23. The method of any one of alternatives 1-22, wherein said enzyme is a dehydrogenase enzyme engineered to bind a non-canonical substrate.
24. The method of alternative 23, wherein said non-canonical substrate comprises one or more alkene unsaturation sites.
25. The method of any one of alternatives 1-24, wherein said enzyme is a desaturase enzyme.
26. The method of alternative 25, wherein said desaturase enzyme is engineered.
27. The method of any one of alternatives 25-26, wherein said desaturase enzyme is engineered to bind a transition metal in its active site.
28. The method of alternative 27, wherein said active site comprises at least 2 cysteine residues that support transition metal binding.
29. The method of any one of alternatives 27-28, wherein the transition metal is nickel, iron, or palladium.
30. The method of any one of alternatives 27-29, wherein said active site comprises an arginine residue that is configured to support a frustrated Lewis pair reaction.

### BRIEF DESCRIPTION OF THE DRAWINGS

In order to describe the manner in which the above-recited and other advantages and features of the embodiments described herein can be obtained, a more particular description will be rendered by reference to specific embodiments thereof which are illustrated in the appended drawings. Understanding that these drawings depict only exemplary embodiments and are not therefore to be considered to be limiting of its scope, the embodiments herein will be described and explained with additional specificity and detail through the use of the accompanying drawings in which:

FIG. 1 depicts an exemplary method for enzymatic biochemical saturation as described in some embodiments herein.

FIG. 2 depicts a ribbon model (left) and a space filled Van der Waals surface model (right) of an example engineered desaturase enzyme. The images depict the molecular docking of tristearin in the active site of desaturase 9, and include the metal atoms coordinated in the active site.

FIG. 3 depicts an exemplary mechanism for H₂ oxidation of fatty acid double bonds by a nickel binding active site. Hydrogenase-like reactions will have the potential to reversibly convert molecular hydrogen into protons and electrons. Furthermore, and given the specific conditions, free protons will have the potential to form hydride ions with the ion metals on site, later promoting the oxidation and consequent reduction of a double carbon bond. For example, when the total number of carbons in the fatty acid is 18, this would represent the conversion of oleic acid into stearic acid.

FIG. 4 depicts a frustrated Lewis pair reaction. In order to improve the oxidation of H₂, a frustrated Lewis pair reaction can be developed in the active site of a desaturase, where the reverse reaction of this enzyme can consequently be performed.

FIG. 5 depicts an exemplary enzyme and active site for alkene hydrogenation. The large subunit of a hydrogenase is shown on the left, shaded by the corresponding hydrophobicity level for each residue (white=less hydrophobic, dark=more hydrophobic). Alkene hydrogenation can occur if the circled region is modified for more hydrophobic residues (right). The recognized alkene can be enzymatically hydrogenated by the catalytic core, with the participation of the corresponding metal nickel-iron core, and arginine 509 (R509).

FIG. 6 depicts an example of enzymatic hydrogenation of a fatty acid, showing a schematic representation of an example mechanism of fatty acid hydrogenation by a modified hydrogenase. The enzymatic reaction starts by the incorporation of molecular hydrogen to the active site, where it is split in two protons by the [Ni—Fe] metal core and an opposite arginine. Each proton can bind to the carbon-carbon double bond section of an oleic acid by proximity, forming stearic acid. The latter is removed and replaced by another oleic acid, and a new reaction starts.

FIG. 7A depicts a schematic representation of a novel enzyme design. FIG. 7B depicts nickel binding properties as defined by fluorescence assay. Nickel binding in a designed pocket greatly reduces the intrinsic protein fluorescence in the protein ID #8 (having an amino acid sequence as set forth in SEQ ID NO: 8) novel enzyme design, but not to the same degree in a design lacking the nickel binding domain (protein ID #6; having an amino acid sequence as set forth in SEQ ID NO: 6).

FIGS. 8A and 8B depict results of desaturation of an engineered desaturase enzyme. FIG. 8A depicts a canonical desaturation of stearic acid as reflected by the increase in the iodine value. FIG. 8B depicts a non-canonical reaction, showing variation of the iodine value of olive oil after five hours of incubation with the desaturase enzyme and hydrogen gas.

FIG. 9 depicts the texture variation of olive oil after treatment with the enzyme described in FIGS. 8A and 8B. The upper fraction of the treated oil (right) presents a spreadable texture.

FIGS. 10A and 10B depict results of enzymatic saturation of free fatty acids with an exemplary enzyme as described herein. Native and novel engineered enzymes were expressed and purified from E. coli and used to saturate fatty acids. FIG. 10A depicts expression of Protein IDs #6-8 (SEQ ID NOs: 6-8) as detected from lysates of E. coli by SDS-PAGE. Asterisks show bands corresponding to proteins of interest. FIG. 10B depicts enzymatic saturation of free fatty acid oil comprised of 90% oleic acid. The oil was saturated using Protein ID #8 (SEQ ID NO: 8) incubated with NiCl₂ and with H₂ bubbling for 8 hours. As a control the same reaction was run with NiCl₂ but without Protein ID #8. The fatty acid composition was analyzed by GC-FID and compared to the untreated oil.

FIGS. 11A-11C depict results of enzymatic hydrogenation of canola oil using metal binding proteins as catalysts. Native and engineered metal binding proteins were expressed and purified from E. coli and used to saturate fatty acids. FIG. 11A depicts expression of Protein ID #40 (SEQ ID NO: 40) and FIG. 11B depicts expression of Protein IDs #41-42 (SEQ ID NOs: 41 and 42) as detected from lysates of E. coli by SDS-PAGE. Asterisks show bands corresponding to proteins of interest. FIG. 11C depicts enzymatic saturation of canola oil using Protein ID #40 incubated with NiCl₂ and with H₂ bubbling for 8 hours. The fatty acid composition was analyzed by GC-FID and compared to the untreated oil.

FIGS. 12A and 12B depict a molecular model representation of a designed metal binding site. FIG. 12A shows protein residues involved in the interaction are labeled, such as histidine, aspartic acid, and glutamic acid. FIG. 12B shows octahedral coordination sites.

FIGS. 13A and 13B depict enzymatic hydrogenation of canola oil using novel protein as catalyst. A native enzyme (Prot.ID #13-SEQ ID NO: 13) was modified to improve it affinity towards free fatty acids, and to contain a metal binding domain (Prot.ID #15-SEQ ID NO: 15), and used to partially saturate canola oil FIG. 13A shows expression of Protein IDs #15 (SEQ ID NO: 15) and #13 (SEQ ID NO: 13) as detected from lysates of E. coli by SDS-PAGE. Asterisks show proteins of interest. S: soluble fraction, I: Insoluble fraction. FIG. 13B shows enzymatic saturation of canola oil using Protein ID #15 (SEQ ID NO: 15) incubated with NiCl₂ and with H₂ bubbling for 8 hours. The fatty acid composition was analyzed by GC-FID and compared to the untreated oil.

### DETAILED DESCRIPTION

Provided herein are methods and processes for the hydrogenation of target molecules containing unique or multiple double or triple atomic bonds. In some embodiments, the methods use one or more protein catalysts (also referred to herein as enzyme catalysts) in soluble suspension or immobilized form together with a hydrogen donor compound to generate the saturation of the target double or triple bonds to obtain a partially or fully saturated product.

In the description that follows, the terms should be given their plain and ordinary meaning when read in light of the specification.

“About” as used herein when referring to a measurable value is meant to encompass variations of ±20% or ±10%, more preferably ±5%, even more preferably ±1%, and still more preferably ±0.1% from the specified value.

As used herein, the term “hydrogenation” has its ordinary meaning as understood in light of the specification, and refers to a reduction reaction wherein hydrogen is the reducing agent. As used herein, the term “saturation,” “saturated,” or “unsaturated” has its ordinary meaning as understood in light of the specification, and refers to a degree of single bonds in a lipid or fatty acid chain. For example, a saturated fatty acid refers to a lipid or fatty acid chain having all or predominantly all single bonds, whereas an unsaturated fatty acid refers to a lipid or fatty acid chain having at least a single double bond. A partially saturated molecule refers to a molecule that has saturated bonds and unsaturated bonds. For example, a partially saturated molecule may include at least one single bond in a lipid or fatty acid.

As used herein, the term “hydrogenase activity” has its ordinary meaning as understood in light of the specification, and refers to an enzyme having an ability to catalyze hydrogenation in a substrate. As used herein, the term “substrate” has its ordinary meaning as understood in light of the specification, and refers to a molecule that specifically binds to a specific binding site in an enzyme, and upon which catalysis takes place.

As used herein the term “molecule” has its ordinary meaning as understood in light of the specification, and refers to a range of compounds including but not restricted to: unsaturated aliphatic hydrocarbon compounds (alkenes, alkynes, alkyl cycloalkenes, cycloalkenes, cycloalkynes, dienes, polyenes, polyynes and their derivatives); aromatic hydrocarbons; heterocyclic aromatic hydrocarbons, polycyclic aromatic hydrocarbons; double bonding organic molecules (aldehydes, amides, carboxylic acids, carboxylate esters, imines, ketones, thioketones, thiols), double bonding inorganic molecules (azo compounds, alkylidenesilanes, disulfurs, germenes, nitroso compounds, plumbenes, silenes, sulfoxides, sulfones, stannenes); triple bonding organic molecules (alkynes, cyanides, isocyanides); mono- and polyunsaturated lipids (including triglycerides, diglycerides, monoglycerides, free fatty acids, phospholipids, waxes, cholesterols and their esters and derivatives). As used herein, “molecule(s)” can refer to the pure compound, or mixtures of these compounds as found in products of animal, vegetable, or synthetic origin.

Examples of unsaturated fatty acids include, for example, stearidonic acid, cervonic acid, myristoleic acid, palmitoleic acid, sapienic acid, oleic acid, elaidic acid, linoleic acid, linoelaidic acid, alpha-linolenic acid, gamma-linolenic acid, dihomo-gamma-linolenic acid, arachidonic acid, erucic acid, docosahexaenoic acid, docosatetraenoic acid, vaccenic acid, paullinic acid, gondoic acid, erucic acid, nervonic acid, mead acid, and eicosapentaenoic acid.

Examples of saturated fatty acids include, for example, propionic acid, butyric acid, valeric acid, caproid acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arichidic acid, heneicosylic acid, behenic acid, tricosylic acid, lignoceric acid, pentacosylic acid, cerotic acid, carboceric acid, montanic acid, nonacosylic acid, melissic acid, hentriacontylic acid, lacceroic acid, psyillic acid, geddic acid, ceroplastic acid, hexatriacontylic acid, heptatriacontylic acid, octatriacontylic acid, nonatriacontylic acid, and tetracontylic acid.

As used herein, the term “alkyl” refers to straight chained and branched saturated hydrocarbon groups. The term Cn means the alkyl group has “n” carbon atoms. For example, C₄ alkyl refers to an alkyl group that has 4 carbon atoms. C₁₋₆ alkyl refers to an alkyl group having a number of carbon atoms encompassing the entire range (i.e., 1 to 6 carbon atoms), as well as all subgroups (e.g., 1-5, 2-5, 1-4, 2-5, 1, 2, 3, 4, 5, and 6 carbon atoms). Unless otherwise indicated, an alkyl group can be an unsubstituted alkyl group or a substituted alkyl group.

As used herein, the term “alkene” is defined identically as “alkyl” except for containing at least one carbon-carbon double bond. Thus, an alkene unsaturation refers to an unsaturated alkene molecule.

In some embodiments, the term “fat” or “fat molecule” refers to a triglyceride, diglyceride, or monoglyceride. The term also may refer to a free fatty acid, phospholipid, or a wax. The term “lipid” can refer to the purified molecule, as well as mixtures of these molecules found in fat products of animal, vegetal or synthetic origin, including oils, lards, butters and tallows.

“Hydrogen donor” has its ordinary meaning as understood in light of the specification, and refers to any compounds that are able to release a hydrogen atom upon interacting with the protein catalysts, including but not limited to hydrogen gas, nucleotidic coenzymes (NADH, NADPH, FADH₂) or donor molecules such as formic acid, isopropanol or dihydroanthracene.

The reaction may also contain, in addition to the protein catalyst, target molecule(s) and hydrogen donor, a suitable polar or nonpolar solvent or mixture thereof. As used herein, the term “solvent” has its ordinary meaning as understood in light of the specification, and refers to a substance capable of at least partially dissolving another substance. Solvents may be liquids at room temperature. Solvents may be organic solvents (for example, having at least one carbon atom) and water. In some embodiments, the solvent may be formed by the combination of two or more organic solvents, or by the combination of an organic solvent and water.

Suitable organic solvents may include, for example, optionally chlorinated aliphatic, cycloaliphatic or aromatic hydrocarbons such as n-pentane, n-heptane, n-octane, cyclopentane, cyclohexane, benzene, toluene, xylenes and chlorobenzene; aromatic, aliphatic and cyclic ethers such as anisole, diethyl ether, di-isopropyl ether, tetrahydrofuran, methyltert-butyl ether and dioxane; N-substituted morpholines, such as N-methylmorpholine and N-formylmorpholine; nitriles, particularly benzonitrile and alkylnitriles having 2 to 5 carbon atoms, such as propionitrile and butyronitrile; 3-methoxypropionitrile and 3-ethoxypropionitrile; dialkyl sulfoxides such as dimethyl and diethyl sulfoxide; N,N-dialkylamides of aliphatic monocarboxylic acids having 1 to 3 carbon atoms in the acid part, such as N,N-dimethylformamide and N,N-dimethylacetamide; alcohols having up to 8 carbon atoms, such as ethanol, n-propanol and tert-butanol; aliphatic and cyclic ketones, such as acetone, diethyl ketone, methyl isopropyl ketone, cyclopentanone, cyclohexanone, 1,3-dimethyl-2-imidazolidinone and 1,3-dimethyl-3,4,5,6-tetrahydro-2-(1H)-pyrimidinone; tetramethylurea; esters, such as esters of carbonic acid, such as diethyl carbonate; nitromethane; alkyl or alkoxyalkyl esters of aliphatic monocarboxylic acids having a total of 2 to 8 carbon atoms, such as methyl, ethyl, n-butyl and isobutyl acetate, ethyl and n-butyl butyrate, and 1-acetoxy-2-ethoxyethane and 1-acetoxy-2-methoxyethane or triethyl phosphate, or chlorinated aliphatic hydrocarbons, such as dichloromethane and chloroform, and diethyl ether, tert-butyl methyl ether, tetrahydrofuran, dioxane, ethanol, n-propanol, acetonitrile and tert-butanol.

The reaction can be performed in conditions that partially or totally remove the presence of oxygen, including vacuum, displacement with other gases, or reaction with oxygen-consuming chemicals; in order to avoid the oxidation of double or triple bonds and the formation of unwanted side-products.

The temperature and pressure conditions of the reaction can be manipulated as well, in order to modulate conversion rates and to as well favor or disfavor the production of specific reaction products.

In a first step of some embodiments of the reaction, an enzyme catalyst hydrolyzes a hydrogen gas or a hydrogen donor molecule and then transfers the free positively charged hydrogen to the double or triple atomic bond of the substrate molecule, reducing the atoms forming this bond.

As used herein, the term “purity,” “pure,” or “purified” has its ordinary meaning as understood in light of the specification and refers to physical separation of a substance of interest from other substances. In certain embodiments, the “purity” of any given agent (e.g., an enzyme or a saturated or unsaturated molecule) in a composition may be specifically defined. For instance, certain compositions may include, for example, an agent that is at least 80, 85, 90, 91, 92, 93, 94, 95, 96, 97, 98, 99, or 100% pure, including all decimals in between, as measured, for example and by no means limiting, by biochemical or analytical methods.

The term “isolated” is meant material that is substantially or essentially free from components that normally accompany it in its native state.

The term oil refers to a lipid composition, which can include the aforementioned molecules, in different proportions, where the melting temperature is lower than the ambient temperature, causing it to be in a liquid state, while the term fat, refers to compounds, which at room temperature are in a solid state.

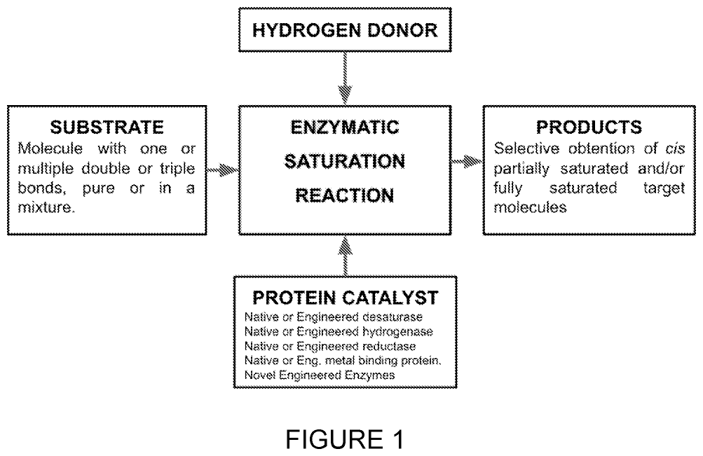

An exemplary method for saturating an unsaturated molecule using embodiments of the methods provided herein is summarized in FIG. 1. In some embodiments, the reaction may be carried out in a reactor containing a substrate lipid with one or more unsaturated molecules (carbon-carbon double bonds) in a suitable solvent mixed with hydrogen gas or a hydrogen donor molecule (i.e., NADH, NADPH or FADH₂). The reaction may also contain an enzyme catalyst in a suitable solvent.

In some embodiments, the methods are carried out as described in FIG. 1. In some embodiments, a first step includes providing a substrate as a starting material, which may include a molecule with one or multiple double or triple bonds. In some embodiments, the starting material is pure or in mixed form. The substrate may then be subjected to an enzymatic saturation reaction, which includes contact with an enzyme catalyst as described and provided herein with a hydrogen donor. In some embodiments, the enzyme catalyst (also referred to herein as a protein catalyst) is a native or engineered enzyme. In some embodiments, the enzyme catalyst is a desaturase, a hydrogenase, a reductase, a metal binding protein, or a novel engineered enzyme. In some embodiments, the hydrogen donor is a hydrogen gas. In some embodiments, the enzymatic saturation reaction includes transfer of a free positively charged hydrogen to a carbon-carbon double bond of the substrate (starting material), thereby producing a saturated carbon-carbon bond, resulting in a product. This step can be carried out at temperature and pressure conditions that can be higher or lower than normal room conditions, in order to improve the catalytic efficiency of the process, while preserving the function of the enzyme.

In some embodiments, the reaction is carried out at a temperature ranging from about −10° C. to about 100° C., such as −10, −5, 0, 5, 10, 15, 20, 25, 30, 35, 40, 45, 50, 55, 60, 65, 70, 75, 80, 85, 90, 95, or 100° C., or at a temperature within a range defined by any two of the aforementioned values. In some embodiments, the reaction is carried out at a pressure ranging from about 0.5 atm to about 5 atm, such as 0.5, 1, 1.5, 2, 2.5, 3, 3.5, 4, 4.5, or 5 atm, or at a pressure within a range defined by any two of the aforementioned values. In some embodiments, the reaction is carried out for a time period ranging from about 0.1 hours to about 24 hours, such as 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 5.5, 6, 7, 8, 9, 10, 11, 12, 13, 14, 15, 16, 17, 18, 19, 20, 21, 22, 23, or 24 hours, or for a period of time within a range defined by any two of the aforementioned values.

In the second step of some embodiments, the product is separated, thereby recovering at least a partially saturated lipid. In some embodiments, the product includes selective obtention of cis partially saturated and/or fully saturated target molecules.

In some embodiments, the reaction may be carried out in a continuous flux reactor containing the substrate lipid in a suitable solvent mixed with hydrogen or a hydrogen donor molecule and the enzyme catalyst may be embedded in a solid-state polymer, glass matrix or any type of immobilization surface. In this case the enzyme can be reutilized multiple times.

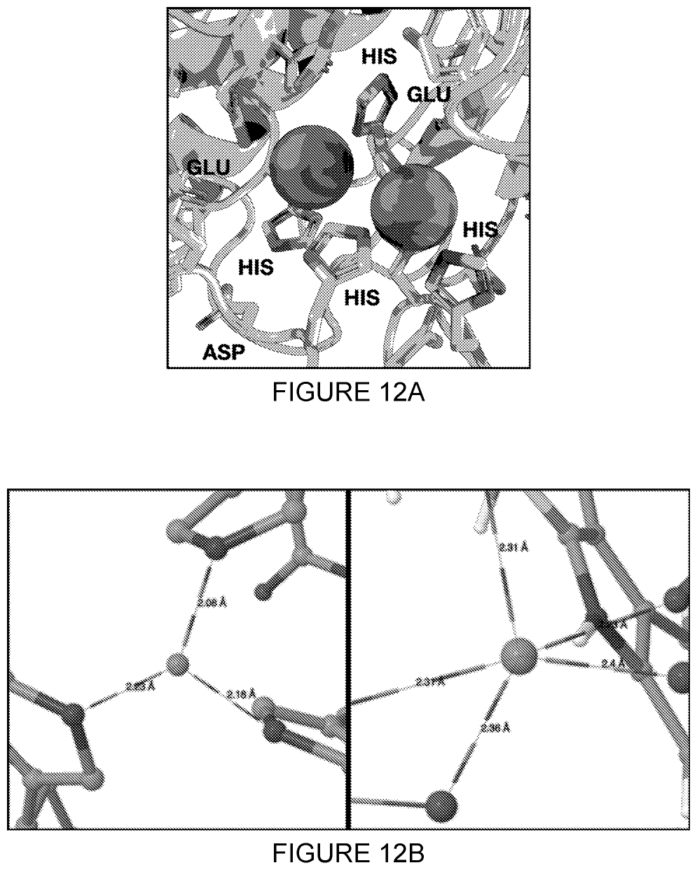

In some embodiments, the enzyme catalyst is an engineered metal binding protein. In some embodiments, the enzyme catalyst contains a metal binding site and/or domain, such as nickel, iron, palladium, or any other transition metal. In some embodiments, the metal binding site is coordinated in an octahedral or tetrahedral coordination geometry (as shown in FIG. 12B), with amino acids such as histidine, cysteine, glutamic acid, and/or aspartic acid, allowing the oxidation of molecular hydrogen (H₂) in order to reduce lipid molecules in a mixture (as shown in FIG. 12A).

In some embodiments, the metal binding site includes a nickel binding motif in the form of His-Xaa(4)-Asp-His (SEQ ID NO: 53). Table 1 depicts various engineered and natural enzymes that may be used in the methods described herein.

TABLE 1

| SEQ | |
|---|---|
| ID | |
| NO | Sequence |
| 1 | VISYDNYVTILDEETLKAWIAKLEKAPVFAFDTET |
| | DSLDNISANLVGLSFAIEPGVAAYIPVAHDYLDAP |
| | DQISRERALELLKPLLEDEKALKVGQNLKYDRGIL |
| | ANYGIELRGIAFDTMLESYILNSVAGRHDMDSLAE |
| | RWLKHKTITFEEIAGKGKNQLTFNQIALEEAGRYA |
| | AEDADVTLQLHLKMWPDLQKHKGPLNVFENIEMPL |
| | VPVLSRIER |
| 2 | VISYDNYVTILDEETLKAWIAKAEKAPVFAFDTHT |
| | DSLDNISANLVGLSFAIEPGVAAYIPVAHDYLDAP |
| | DQISRERALELLKPLLEDEKALKVGHELKHDRGIL |
| | ANYGIELRGIAFDTMLESYILNSVAGRHDMDSLAE |
| | RWLKHKTITFEEIAGKGKNQLTFNQIALEEAGRHA |
| | AEHADVTLQLHLKMWPDLQKHKGPLNVFENIEMPL |
| | VPVLSRIER |
| 3 | VISYDNYVTILDEETLKAWIAKAEKAPVFAFDTCT |
| | CCCSNISALGCNVLCLIEPGVAAYIPVAHDYLDAP |
| | DQISRERALELLKPLLEDEKALKCHIELKLCRDGI |
| | CNYGIELRGIAFCCIFESYILNSVAGRHCCCCLAE |
| | RWLKHKTITFEEIAGKGKNQLTFCQIALEEAGRHA |
| | CDCARHTLQLHLKMWPDLQKHKGPLNVFENIEMPL |
| | VPVLSRIER |
| 4 | VISYDNYVTILDEETLKAWIAKAEKAPVFAHDHHT |
| | DSLDNISANLVGLSFAIEPGVAAYIPVAHDYLDAP |
| | DQISRERALELLKPLLEDEKALKVGHELKHDRGIL |
| | ANYGIELRGIAFDTMLESYILNSVAGRHDMDSLAE |
| | RWLKHKTITFEEIAGKGKNQLTFNQIALEEAGRHA |
| | AEHADVTLQLHLKMWPDLQKHKGPLNVFENIEMPL |
| | VPVLSRIER |
| 5 | VISYDNYVTILDEETLKAWIAKAEKAPVFADCHCT |
| | FEQNNISAMKCCQAICIEPGVAAYIPVAHDYLDAP |
| | DQISRERALELLKPLLEDEKALKHCCELPFFRCCC |
| | MNYGIELRGIAFYCFCESYILNSVAGRHAWRCLAE |
| | RWLKHKTITFEEIAGKGKNQLTACQIALEEAGRHA |
| | CCCAVYTLQLHLKMWPDLQKHKGPLNVFENIEMPL |
| | VPVLSRIER |
| 6 | AFWGASNELLHDPTMIKEGSSWYALGTGLTEERGL |
| | RVLKSSDAKNWTVQKSIFTTPLSWWSNYVPNYGQN |
| | QWAPDIQYYNGKYWLYYSVSSFGSNTSAIGLASST |
| | SISSGGWKDEGLVIRSTSSNNYNAIDPELTFDKDG |
| | NPWLAFGSFWSGIKLTKLDKSTMKPTGSLYSIAAR |
| | PNNGGALEAPTLTYQNGYYYLMVSFDKCCDGVNST |
| | YKIAYGRSKSITGPYLDKSGKSMLEGGGTILDSGN |
| | DQWKGPGGQDIVNGNILVRHAYDANDNGIPKLLIN |
| | DLNWSSGWPSY |
| 7 | AFWGASNELLHDPTMIKEGSSWYALGTGLTEERGL |
| | RVLKSSDAKNWTVQKSIFTTPLSWWSNYVPNYGQN |
| | QWRPDIQYYNGKYWLYYSVSSFGSNTSAIGLASST |
| | SISSGGWKDEGLVIRSTSSNNYDAEDPELTFDKDG |
| | NPWLAFGDFRSGIKLTKLDKSTMKPTGSLYSIAAR |
| | PNNGGALEAPTLTYQNGYYYLMVSFDKCCDGVNST |
| | YKIAYGRSKSITGPYLDKSGKSMLEGGGTILDSGN |
| | DQWKGPGGQDIVNGNILVRHAYDANDNGIPKLLIN |
| | DLNWSSGWPSY |
| 8 | AFWGASNELLHDPTMIKEGSSWYALMTGLTEERGL |
| | RVLKSSDAKNWTVQKSIFTTPLSWWSNYVPKYGQN |
| | QWRPDIQYYNGKYWLYYSVWSFGSNTSAIGLASST |
| | SISSGGWKDEGLVIYSTSSNNYDAEDDELTFDKDG |
| | NPWLAFGDFRSGIKLTKLDKSTMKPTGSLYSIAMR |
| | PNNGGALEAPTLTYQNGYYYLMVSFFKCCWGVNFT |
| | YKIAYGRSKSITGPYLDKSGKSMLEGGGTILDSGN |
| | DQWKGPGGQDIVNGNILVRHAYDANDNGIPKLLIN |
| | DLNWSSGWPSY |
| 9 | AFWGASNELLHDPTMIKEGSSWYALGTGLTEERGL |
| | RVLKSSDAKNWTVQKSIFTTPLSWWSNYVPNYGQN |
| | QHAPDIQYYNGKYWLYYSVESFGSNTSAIGLASST |
| | SISSGGWKDEGLVIRSTSSNNYHADHPELTFDKDG |
| | NPWLAFGHHESGIKLTKLDKSTMKPTGSLYSIAAR |
| | PNNGGALHAPTLTYQNGYYYLMVSFDKCCDGVNST |
| | YKIAYGRSKSITGPYLDKSGKSMLEGGGTILDSGN |
| | DQWKGPGGQDIVNGNILVRHAYDANDNGIPKLLIN |
| | DLNWSSGWPSY |
| 10 | AFWGASNELLHDPTMIKEGSSWYALGTGLTEERGL |
| | RVLKSSDAKNWTVQKSIFTTPLSWWSNYVPNYGQN |
| | QHAPDIQYYNGKYWLYYSVESCGCKCCVIGLASST |
| | SISSGGWKDEGLVIRSTSSNNYLACHPELTFDKDG |
| | NPWLAFGCHESCCCLTKLDKSTMKPTGSLYSIAAR |
| | PNNGGNSVACTLTYQNGYYYLMVSFDKCCDGVCCA |
| | CKIAYGRSKSITGPYLDKSGKSMLEGGGTILDSGN |
| | DQWKGPGGQDIVNGNILVRHAYDANDNGIPKLLIN |
| | DLNWSSGWPSY |
| 11 | AFWGASNELLHDPTMIKEGSSWYALGTGLTEERGL |
| | RVLKSSDAKNWTVQKSIFTTPLSWWSNYVPNYGQN |
| | QHAPDIQYYNGKYWLYYSVESFGSNTSAIGLASST |
| | SISSGGWKDEGLVIRSTSSNNYHACHPELTFDKDG |
| | NPWLAFGCCESGIKLTKLDKSTMKPTGSLYSIAAR |
| | PNNGGALDAPTLTYQNGYYYLMVSFEKCCDGVRST |
| | YKIAYGRSKSITGPYLDKSGKSMLEGGGTILDSGN |
| | DQWKGPGGQDIVNGNILVRHAYDANDNGIPKLLIN |
| | DLNWSSGWPSY |
| 12 | AFWGASNELLHDPTMIKEGSSWYALGTGLTEERGL |
| | RVLKSSDAKNWTVQKSIFTTPLSWWSNYVPNYGQN |
| | QHAPDIQYYNGKYWLYYSCRSYCCCSCCIGLASST |
| | SISSGGWKDEGLVIRSTSSNNYHACHPELTFDKDG |
| | NPWLAFGCCESCCCLTKLDKSTMKPTGSLYSIAAR |
| | PNNGCCCCACTLTYQNGYYYLMVSFEKCCDGVYSC |
| | CKIAYGRSKSITGPYLDKSGKSMLEGGGTILDSGN |
| | DQWKGPGGQDIVNGNILVRHAYDANDNGIPKLLIN |
| | DLNWSSGWPSY |
| 13 | MNYLNNIRIENPLTICYTNDVVKNFTANGLLSIGA |
| | SPAMSEAPEEAEEFYKVAQALLINIGTLTAQNEQD |
| | IIAIAQTANEAGLPIVFDPVAVGASTYRKQFCKLL |
| | LKSAKVSVIKGNASEILALIDDTATLDAVTIAKKA |
| | YAIYKTAIVITGKEDVIVQGDKAIVLANGSPLLAR |
| | VTGAGCLLGGIIAGFLFRETEPDIEALIEAVSVFN |
| | IAAEVAAENENCGGPGTFSPLLLDTLYHLNETTYQ |
| | QRIRIQEVE |
| 14 | MNYLNNIRIENPLTICYTNDVVKNFTANGLLSIGA |
| | SPAMSEAPEEAEEFYKVAQALLINIGTLTAQNEQD |
| | IIAIAQTANEAGLPIVFDPVAVGASTYRKQFCKLL |
| | LKSAKVSVIKGNASEILALIDDTATLDAVTIAKKA |
| | YAIYKTAIVIHGKEDVIVQGDKAIVLANGSPLLAR |
| | VTGAGCLLGGIIAGFLFRETEPDIEALIEAESVHH |
| | IAAEVAAENENCGGPGTFSPLLLDTLYHLNETTYQ |
| | QRIRIQEVE |
| 15 | MNYLNNIRIENPLTICYTNDVVKNFTANGLLSIGA |
| | SPAMSEAPEEAEEFYKVAQALLINIGTLTAQNEQD |
| | IIAIAQTANEAGLPIVFDPVAVGASTYRKQFCKLL |
| | LKSAKVSVIKCIYNEILALIDDTATLDAVTIAKKA |
| | YAIYKTAIVPMGKEVVSWQGDKAIHFDPPMPSHFP |
| | VTGAGCLGMTISAGFLFRETEPDIEALDTFCSCCH |
| | IYGCEAAENENCGGPGTFSPLLLDTLYHLNETTYQ |
| | QRIRIQEVE |
| 16 | MNYLNNIRIENPLTICYTNDVVKNFTANGLLSIGA |
| | SPAMSEAPEEAEEFYKVAQALLINIGTLTAQNEQD |
| | IIAIAQTANEAGLPIVFDPVAVGASTYRKQFCKLL |
| | LKSAKVSVIKGNASEILALIDDTATLDAVTIAKKA |
| | YAIYKTAIVIHGKEHVIVQGDKAIVLANGSPLEAR |
| | VTGAGCLHGGIIAGFLFRETEPDIEALIEAHSVHH |
| | IAAEVAAENENCGGPGTFSPLLLDTLYHLNETTYQ |
| | QRIRIQEVE |
| 17 | MNYLNNIRIENPLTICYTNDVVKNFTANGLLSIGA |
| | SPAMSEAPEEAEEFYKVAQALLINIGTLTAQNEQD |
| | IIAIAQTANEAGLPIVFDPVAVGASTYRKQFCKLL |
| | LKSAKVSVIKAKDMEILALIDDTATLDAVTIAKKA |
| | YAIYKTAIVKYGCGCVSKQGDKAICCCEGRWKQAR |
| | VTGCACDFGNLIAGFLFRETEPDIEALECCCSMWH |
| | IQCWEAAENENCGGPGTFSPLLLDTLYHLNETTYQ |
| | QRIRIQEVE |
| 18 | MNYLNNIRIENPLTICYTNDVVKNFTANGLLSIGA |
| | SPAMSEAPEEAEEFYKVAQALLINIGTLTAQNEQD |
| | IIAIAQTANEAGLPIVFDPVAVGASTYRKQFCKLL |
| | LKSAKVSVIKGNASEILALIDDTATLDAVTIAKKA |
| | YAIYKTAIVICGKECVIVQGDKAIVLANGSPLEAR |
| | VTGAGCLCGGIIAGFLFRETEPDIEALIEAHSVHC |
| | IAAEVAAENENCGGPGTFSPLLLDTLYHLNETTYQ |
| | QRIRIQEVE |
| 19 | MNYLNNIRIENPLTICYTNDVVKNFTANGLLSIGA |
| | SPAMSEAPEEAEEFYKVAQALLINIGTLTAQNEQD |
| | IIAIAQTANEAGLPIVFDPVAVGASTYRKQFCKLL |
| | LKSAKVSVIKVQCPEILALIDDTATLDAVTIAKKA |
| | YAIYKTAIVICGWSCVHQQGDKAIIVCKGHMTAWK |
| | VTGLWCLCGSMVCGFLFRETEPDIEALEGCISHIC |
| | ICVKKAAENENCGGPGTFSPLLLDTLYHLNETTYQ |
| | QRIRIQEVE |
| 20 | MNYLNNIRIENPLTICYTNDVVKNFTANGLLSIGA |
| | SPAMSEAPEEAEEFYKVAQALLINIGTLTAQNEQD |
| | IIAIAQTANEAGLPIVFDPVAVGASTYRKQFCKLL |
| | LKSAKVSVIKGNASEILALIDDTATLDAVTIAKKA |
| | YAIYKTAIVIITEGDVIVQGDKAIVLANGSPLLAR |
| | VTGAGCLHGGIIAGFLFRETEPDIEALIEACSVSE |
| | IAAEVAAENENCGGPGTFSPLLLDTLYHLNETTYQ |
| | QRIRIQEVE |
| 21 | MNYLNNIRIENPLTICYTNDVVKNFTANGLLSIGA |
| | SPAMSEAPEEAEEFYKVAQALLINIGTLTAQNEQD |
| | IIAIAQTANEAGLPIVFDPVAVGASTYRKQFCKLL |
| | LKSAKVSVIKGNASEILALIDDTATLDAVTIAKKA |
| | YAIYKTAIVIIEEGDVIVQGDKAIVLANGSPLLAR |
| | VTGAGCLHGGIIAGFLFRETEPDIEALIEACSVIE |
| | IAAEVAAENENCGGPGTFSPLLLDTLYHLNETTYQ |
| | QRIRIQEVE |
| 22 | MSALTDLFPLPIVQAPMAGGVSVPQLAAAVCEAGG |
| | LGFLAAGYKTADGMYQEIKRLRGLTGRPFGVNVFM |
| | PQPELGAVEVYAHQLAGEAAWYETELGDPDGGRDD |
| | GYDAKLAVLLDDPVPVVSFHFGVPDREVIARLRRA |
| | GTLTLVTATTPEEARAVEAAGADAVIAQGVEAGGH |
| | QGTHRDSSEDDGAGIGLLSLLAQVREAVDIPVVAA |
| | GGIMRGGQIAAVLAAGADAAQLGTAFLATDESGAP |
| | GPHKRALTDPLFARTRLTRAFTGRPARSLVNRFLR |
| | EHGPYAPAAYPDVHHLTSPLRKAAAKAGDAQGMAL |
| | WAGQGHRMARELPAGRLVEVLAAELAEARTALS |
| 23 | MSALTDLFPLPIVQHDHHGGVSVPQLAAAVCEAGG |
| | LGFLAAGYKTADGMYQEIKRLRGLTGRPFGVDVHM |
| | PQPELGAVEVYAHQLAGEAAWYETELGDPDGGRDD |
| | GYDAKLAVLLDDPVPVVSFHFGVPDREVIARLRRA |
| | GTLTLVTATTPEEARAVEAAGADAVIAQGVEAGGH |
| | QGTHRDSSEDDGAGIGLLSLLAQVREAVDIPVVAA |
| | GGIMRGGQIAAVLAAGADAARLGTAFLATDESGAP |
| | GPHKRALTDPLFARTRLTRAFTGRPARSLVNRFLR |
| | EHGPYAPAAYPDVHHLTSPLRKAAAKAGDAQGMAL |
| | WAGQGHRMARELPAGRLVEVLAAELAEARTALS |
| 24 | MSALTDLFPLPIVQHDHHLGASVPQLAAAVCEAGG |
| | LGFLAARYKTADGMYQEIKRLRGLTGRFFGVDVHM |
| | PQPELGAVEVYAHQLAGEAAWYETELGDPDGGRDD |
| | GYDAKLAVLLDDPVPVVSFHFGVPDREVIARLRRA |
| | GTWTLFTATTPEEARAVEAAGADAVIAQGVTAGGH |
| | QGSHRDSSEDDGAGIGLLSLLAQVREAVDIPVVAA |
| | GGTMRGGQIAALLAAGFDAARLGTAFLATDESGAP |
| | GPHKRALTDPLFARTRLTRAFTGRPARSLVNRFLR |
| | EHGPYAPAAYPDVHHLTQPLRKAWYKAGDAQGMAL |
| | WAGQGHRMARELPAGRLVEVLAAELAEARTALS |
| 25 | MQTFVHEAGRLPAHIAAELGSYRYRVFVEQLGWQL |
| | PSEDEKMERDQYDRDDTVYVLGRDANGEICGCARL |
| | LPTTRPYLLQEVFPHLLADEAPRSAHVWELSRFAA |
| | TAWSVRPMLAAAVECAARRGARQLIGVTFCSMERM |
| | FRRIGVHAHRAGAPVSIDGRMVVACWIDIDAQTLA |
| | ALDLDPALC |
| 26 | MQTFVHEAGRLPAHIAAELGSYRYRVFVEQLGRQL |
| | PSEDEKMERDQYDRDDTVYVLGRDANGEICGCARL |
| | LPTTRPYLLQEVEPHELADEAPRSAHVWELSRFAA |
| | TAWSVRPMLAAAVECAARRGARQLIGHTDCSMERM |
| | FRRIGVHAHRAGAPVSEDGRMEVACWIDIDAQTLA |
| | ALDLDPALC |
| 27 | MQTFVHEAGRLPAHIAAELGSYRYQNFCSAQGRQL |
| | PSEDEKMERDQYDRDDTVYVLGRDANGEICGCYSR |
| | YPTTRPKDEVEFEPHEFADEAPRSAHVFPVSIFAW |
| | TAWSVRPMLAAAVECAARRGARQLIGDTECRMERM |
| | FRRIGVHAHRAGAGVVEEYRTEFICWIDIDAQTLA |
| | ALDLDPALC |
| 28 | MQPDPIAADILRKEPEQETFVRLNVPLEVPTSDEV |
| | EAYKCLQECLELRKRYVFQETVAPWEKEEPFAHYP |
| | QGKSDHCFEMQDGVVHVFANKDAKEDLFPVADATA |
| | FFTDLHHVLKVIAAGNIRTLCHRRLVLLEQKFNLH |
| | LMLNADKEFLAQKSAPHRDFYNVRKVDTHVHHSAC |
| | RFLGEITKQVFSDLEASKYQMAEYRISIYGRKMSE |
| | WDQLASWIVNNDLYSENVVWLIQLPRLYNIYKDMG |
| | IVTSFQNILDNIFIPLFEATVDPDSHPQLHVFLKQ |
| | VVGFDLVDDESKPERRPTKHMPTPAQWTNAFNPAF |
| | SYYVYYCYANLYVLNKLRESKGMTTITLRPHSGEA |
| | GDIDHLAATFLTCHSIAHGINLRKSPVLQYLYYLA |
| | QIGLAMSPLSNNSLFLDYHRNPFPVFFLRGLNVSL |
| | STDDPLQIHLTKEPLVEEYSIAASVWKLSACDLCE |
| | IARNSVYQSGFSHALKSHWIGKDYYKRGPDGNDIH |
| | KTNVPHIRVEFRDTIWKEEMQQVYLGKAVISDEVE |
| | H |
| 29 | MQPDPIAADILRKEPEQETFVRLNVPLEVPTSDEV |
| | EAYKCLQECLELRKRYVFQETVAPWEKEEPFAHYP |
| | QGKSDHCFEMQDGVVHVFANKDAKEDLFPVADATA |
| | FFTDLHHVLKVIAAGNIRTLCHRRLVLLEQKFNLH |
| | LMLNADKEFLAQKSAPHRDFYNVRKVDTHVHHSAC |
| | RFLGEITKQVFSDLEASKYQMAEYRISIYGRKMSE |
| | WDQLASWIVNNDLYSENVVWLIQLPRLYNIYKDMG |
| | IVTSFQNILDNIFIPLFEATVDPDSHPQLHVFLKQ |
| | VVGFDLVDDESKPERRPTKHMPTPAQWTNAFNPAF |
| | SYYVYYCYANLYVLNKLRESKGMTTITLRPHSGEA |
| | GDIDHLAATFLTCHSIAHGINLRKSPVLQYLYYLA |
| | EIGLAMSPLSNNSLFLDYHRNPFPVFFLRGLHVSL |
| | STDDPLQIHLTKEPLVEEYSIAASVWKLSACDLCE |
| | IARESVHHSGFSHALKSHWIGKDYYKRGPDGNDIH |
| | KTNVPHIRVEFRDTIWKEEMQQVYLGKAVISDEVE |
| | H |
| 30 | MQPDPIAADILRKEPEQETFVRLNVPLEVPTSDEV |
| | EAYKCLQECAELMKRYVFQETVAPWEKEEPFAHYP |
| | QGKSDHCFEMQDGVVHVFANKDAKEDLFPVADATA |
| | FFTDLHHVLKVIAAGNIRTLCHRRLVLLEQKFNLH |
| | LMLNADKEFLAQKSAPHRDFYNVRKVDTHVHHSAC |
| | RFLGEITKQVFSDLEASKYQMAEYRISIYGRKMSE |
| | WDQLASWIVNNDLYSENVVWLIQLPRLYNIYKDMG |
| | IVTSFQNILDNIFIPLFEATVDPDSHPQLHVFLKQ |
| | VVGFDLVDDESKPERRPTKHMPTPAQWTNAFNPAF |
| | SYYVYYCYANLYVLNKLRESKGMTTITCRPHSGEA |
| | GDIDHLAATFLTCVSIAHGINLRKSPVLQYLYYLA |
| | EIFLAKSPLSNNSLFLDYHRNPFPVFFLRGLHVSC |
| | STDDPLQIHLTKEPLVEEYSIAASVWKLSACDLCE |
| | IIRESVHHSGFSHALFSHWIGKDYYQRGPDGNDIH |
| | KTNVPHIRVEFRDTIWKEEMQQVYLGKAVISDEVE |
| | H |
| 31 | MLPNTGRLAGCTVFITGASRGIGKAIALKAAKDGA |
| | NIVIAAKTAQPHPKLLGTIYTAAEEIEAVGGKALP |
| | CIVDVRDEQQISAAVEKAIKKFGGIDILVNNASAI |
| | SLTNTLDTPTKRLDLMMNVNTRGTYLASKACIPYL |
| | KKSKVAHILNISPPLNLNPVWFKQHCAYTIAKYGM |
| | SMYVLGMAEEFKGEIAVNALWPKTAIHTAAMDMLG |
| | GIESQCRKVDIIADAAYSIFQKPKSFTGNFVIDEN |
| | ILKEEGIENFDVYAIKPGHPLQPDFFLD |
| 32 | MLPNTGRLAGCTVFITGASRGDGKAIALKAAKDGA |
| | NIVIAAKTAQPHPKLLGTIYTAAEEIEAVGGKALP |
| | CIVDVRDEQQISAAVEKAIKKFGGIDILVNHAEAI |
| | SLTNTLDTPTKRLDLMMNVNTRGTYLASKACIPYL |
| | KKSKVAHILNHSPPLNLNPVWFKQHCAYTIARYGM |
| | SMYVLGMAEEFKGEIAVNALWPKTAIHTAAMDMLG |
| | GIESQCRKVDIIADAAYSIFQKPKSFTGNFVIDEN |
| | ILKEEGIENFDVYAIKPGHPLQPDFFLD |
| 33 | MLPNTGRLAGCTVFITGASRGDGKNIAQKAAKDGA |
| | NIVILAKTAQPHPKLLGTIYTAAEEIEAVGGKALP |
| | CIVDVRDEQQISAAVEKAIKKFGGIDILVNNAEAI |
| | NLTNTLDTPTKRLDLMMWVNTRGPYLASKACIPYL |
| | KKSKVAHILNHSPPLNLNPVWFKQHCAYTIARYGM |
| | SMYVLGMAEEFKGEIAVNAHWPYTAIHTAAMDMCG |
| | GIESQCGKVDIIAWAAESIFQKPKSFTGNFVINEN |
| | ILKEEGIENFDVYAIKPGHPLQPDFFLD |
| 34 | MLSPAVQTFWKWLQEEGVITAKTPVKASVVTEGLG |
| | LVALKDISRNDVILQVPKRLWINPDAVAASEIGRV |
| | CSELKPWLSVILFLIRERSREDSVWKHYFGILPQE |
| | TDSTIYWSEEELQELQGSQLLKTTVSVKEYVKNEC |
| | LKLEQEIILPNKRLFPDPVTLDDFFWAFGILRSRA |
| | FSRLRNENLVVVPMADLINHSAGVTTEDHAYEVKG |
| | AAGLFSWDYLFSLKSPLSVKAGEQVYIQYDLNKSN |
| | AELALDYGFI |
| 35 | MLSPAVQTFWKWLQEEGVITAKTPVKASVVTEGLG |
| | LVALKDISRNDVILQVPKRLWINPDAVAASEIGRV |
| | CSELKPWLSVILFLIRERSREDSVWKHYFGILPQE |
| | TDSTIYWSEEELQELQGSQLLKTTVSVKEYVKNEC |
| | LKLEQEIILPNKRLFPDPVTLDDFFWAFGILRHRA |
| | ESRLRNENLVVVPMADLEEHSAGVTTEDHAYEVKG |
| | AAGLFSWDYLFSLKSPLSVKAGEQVYIQGDLNKSN |
| | AELALDHGFI |
| 36 | MLSPAVQTFWKWLQEEGVITAKTPVKASVVTEGLG |
| | HVALKDISRNDVILQVPKRLWINPDAVAASEIGRV |
| | CSELKPWLSVILFLIREQSKEDSRWKHYFGILPWE |
| | TDKTIYWSEEELQELQGSQLLKTTVSVKEYVKNEC |
| | LKLEQEIILPNKRLFPDPVTLDDFFWAFGMCRHRA |
| | ESRLRNENLVVVPMADLEEHSAGQTAEHHAYEVCG |
| | AAGQNSWDYLFSWTEPLNVKAGFQVYIQGDLIKSN |
| | AELYLDRGFI |
| 37 | MPFPYEFRELNPEEDKLVKANLGAFPTTYVKLGPK |
| | GYMVYRPYLKDAANIYNMPLRPTDVFVASYQRSGT |
| | TMTQELVWLIENDLNFEAAKTYMSLRYIYLDGFMI |
| | YDPEKQEEYNDILPNPENLDMERYLGLEYSSRPGS |
| | SLLAAVPPTEKRFVKTHLPLSLMPPNMLDTVKMVY |
| | LARDPRDVAVSSFHHARLLYLLNKQSNFKDFWEMF |
| | HRGLYTLTPYFEHVKEAWAKRHDPNMLFLFYEDYL |
| | KDLPGCIARIADFLGKKLSEEQIQRLCEHLNFEKF |
| | KNNGAVNMEDYREIGILADGEHFIRKGKAGCWRDY |
| | FDEEMTKQAEKWIKDNLKDTDLRYPNM |
| 38 | MPFPYEFRELNPEEDKLVKANLGAEPTTYVKLGPK |
| | GYMVYRPYLKDAANIYNMPLRPTDVFVASYQRSGT |
| | TMTQELVWLIENDLNFEAAKTYMSLRYIYLDGFMR |
| | YDPEKQEEYNDELPNPENLDMERYLGLEYSSRPGS |
| | SLLAAVPPTEKRFVKTHLPLSLMPPNMLDTVKMVY |
| | LARDPRDVAVSSFHHARLDYELNKQSNFKDFWEMF |
| | HRGLYTLTPYFEHVKEAWAKRHDPNMLFLFYEDYL |
| | KDLPGCIARIADFLGKKLSEEQIQRLCEHLNFEKF |
| | KNNGAVNMEDEREIGHEADGEHFIRKGKAGCWRDY |
| | FDEEMTKQAEKWIKDNLKDTDLRYPNM |
| 39 | MPFPYEFRELNPEEDKLVKANLDSEPDTEVKLGPK |
| | GYMVYFPDLKDAANIYNMPLRPTDVFVASYQRTGT |
| | TMTQELVWLIENDLNFEAAKTYMSLRYIYDDGFMR |
| | YFPEKLEAYNFELMIPRKPMSEKRYFLEGTVCIGS |
| | SLLAAVPPTEKRFVKTDLELSLMPPNMLDTVKMVY |
| | LARDPRDVAVSSFGNYRRDYSLCIQSNFKDFWEMF |
| | HRGLYTLVPYFEHVKEAWAKRHDPNMLFLFYEDYL |
| | KDLPGCIARIADFLGKKLSEEQIQRLCEHLNFEKF |
| | KNNGAVNMHDTRGQGVEADMIHRISKGKAGCWRDY |
| | FDEEMTKQAEKWIKDNLKDTDLRYPNM |
| 40 | MKIPKDTLIIAVENEIARINPAYSEDHDAVINLVF |
| | SGLTRFDENMSLKPDLAKSWDISKDGLVYDIFLRD |
| | DVLWHDGVKFSADDVKFSIEAFKNPKNNSSIYVNF |
| | EDIKSVEILNPSHVKITLFKPYPAFLDALSIGMLP |
| | KHLLENENLNTSSFNQNPIGTGPYKFVKWKKGEYV |
| | EFKANEHFYLDKVKTPRLIIKHIFDPSIASAELKN |
| | GKIDAALIDVSLLNIFKNDENFGILREKSADYRAL |
| | MFNLDNEFLKDLKVRQALNYAVDKESIVKNLLHDY |
| | AFVANHPLERSWANSKNFKIYKYDPKKAEDLLVSA |
| | GFKKNKDGNFEKDGKILEFEIWAMSNDPLRVSLAG |
| | ILQSEFRKIGVVSKVVAKPAGSFDYSKVDSFLIGW |
| | GSPLDPDFHTFRVFESSQDSALNDEGWNFGHYHDK |
| | KVDIALQKARNTSNLEERKKYYKDFIDALYENPPF |
| | IFLAYLDFALVYNKDLKGIKTRTLGHHGVGFTWNV |
| | YEWSK |
| 41 | MKIPKDTLIIAVENEIARINPAYSEDHDAVINLVF |
| | SGLTRFDENMSLKPDLAKSWDISKDGLVYDIFLRD |
| | DVLWHDGVKFSADDVKFSIEAFKNPKNNSSIYVNF |
| | EDIKSVEILNPSHVKITLFKPYPAFLDALSIGMLP |
| | KHLLENENLNTSSFNQNPIGTGPYKFVKWKKGEYV |
| | EFKANEHFYLDKVKTPRLIIKHIFDPSIASAELKN |
| | GKIDAALIDVSLLNIFKNDENFGILREKSADYRAL |
| | MFNLDNEFLKDLKVRQALNYAVDKESIVKNLLHDY |
| | AFVANHPLERSWANSKNFKIYKYDPKKAEDLLVSA |
| | GFKKNKDGNFEKDGKILEFEIWAMSNDPLRVSLAG |
| | ILQSEFRKIGVVSKVVAKPAGSFDYSKVDSFLEGH |
| | GSPLDPDFHTFRVFESSQDSALNDEGENRGHYHDK |
| | KVDIALQKARNTSNLEERKKYYKDFIDALYENPPF |
| | IFLAYLDFALVYNKDLKGIKTRTLGHHGVGFTWNV |
| | YEWSK |
| 42 | MKIPKDTLIIAVVNEPSRINPAYSWDHDCKANLVF |
| | SGLTRFDENMSLKPDLAKSWDISKDGLVYDIFLRD |
| | DVLWHDGVKFSADDVKFSIEAFKNPKNNSTQCKWA |
| | EDIKSVEILNPSHVKITLFKPYPAFLDALSIGMLP |
| | KHLLENENLNTSSFNQNPIGTGPYKFVKWKKGEYV |
| | EFKANEHFYLDKVKTPRLIICEMNQMFIASAELKN |
| | GKIDAALIDVSLLNIFKNDENFGILREKSADYRAL |
| | MFNLDNEFLKDLKVRQALNYAVDKESIVKNLLHDY |
| | AFVANHPLERSWANSKNFKIYKYDPKKAEDLLVSA |
| | GFKKNKDGNFEKDGKILEFEIWQHSADPLRVSLAG |
| | ILQSEFRKIGVVSKVVAKPRCVFDGKCVDSFLEGH |
| | GSPLDPDFHTFRVFESSQDSALNDDDANYGSDTDK |
| | KVDIALQKARNTSNLEERKKYYKDFIDALYENPPF |
| | IFLAYLDFALVYNKDLKGIKTRTLGHHGVGFTWNV |
| | YEWSK |
| 43 | MALKFNPLVSQPYKLASSARPPVSTFRSPKFLCLA |
| | SSSSPALSSKEVESLKKPFTPPREVHLQVLHSMPP |
| | QKIEIFKSMEDWAEQNLLPHLKDVEKSWQPQDFLP |
| | DPASDGFEDQVKELRERARELPDDYFVVLVGDMIT |
| | EEALPTYQTMLNTLDGVRDETGASPTSWAVWTRAW |
| | TAEENRHGDLLNKYLYLSGRVDMRQIEKTIQYLIG |
| | SGMDPRTENNPYLGFIYTSFQERATFVSHGNTARQ |
| | AKEHGDLKLAQICGTIAADEKRHETAYTKIVEKLL |
| | EIDPDGTVVAFADMMRKKISMPAHLMYDGRDDNLF |
| | DNFSSVAQRLGVYTAKDYADILEFLVGRWRIESLT |
| | GLSGEGNKAQEYLCGLTPRIRRLDERAQARAKKGP |
| | KIPFSWIHDREVQLVD |
| 44 | MPAHLLQEEISSSYTTTTTITAPPSRVLQNGGGKL |
| | EKTPLYLEEDIRPEMRDDIYDPTYQDKEGPKPKLE |
| | YVWRNIILMSLLHLGALYGITLIPTCKIYTYIWVL |
| | FYYLMGALGITAGAHRLWSHRTYKARLPLRVFLII |
| | GNTMAFQNDVFEWSRDHRAHHKFSETDADPHNSRR |
| | GFFFSHVGWLLVRKHPAVKEKGSTLNLSDLRAEKL |
| | VMFQRRYYKPGVLLLCFILPTLVPWYLWDETFQNS |
| | LFFATLFRYALGLNVTWLVNSAAHMYGYRPYDKTI |
| | NPRENILVSLGAAGEGFHNYHHTFPYDYSASEYRW |
| | HINFTTFFIDCMAAIGLAYDRKKVSKAAILARIKR |
| | TGEESYKSGVD |
| 45 | MALKLNPFLSQTQKLPSFALPPMASTRSPKFYMAS |
| | TLKSGSKEVENLKKPFMPPREVHVQVTHSMPPQKI |
| | EIFKSLDNWAEENILVHLKPVEKCWQPQDFLPDPA |
| | SDGFDEQVRELRERAKEIPDDYFVVLVGDMITEEA |
| | LPTYQTMLNTLDGVRDETGASPTSWAIWTRAWTAE |
| | ENRHGDLLNKYLYLSGRVDMRQIEKTIQYLIGSGM |
| | DPRTENSPYLGFIYTSFQERATFISHGNTARQAKE |
| | HGDIKLAQICGTIAADEKRHETAYTKIVEKLFEID |
| | PDGTVLAFADMMRKKISMPAHLMYDGRDDNLFDHF |
| | SAVAQRLGVYTAKDYADILEFLVGRWKVDKLTGLS |
| | AEGQKAQDYVCRLPPRIRRLEERAQGRAKEAPTMP |
| | FSWIFDRQVKLVD |
| 46 | MALKLCFPPHKMPSFPDARIRSHRVFMASTIHSPS |
| | MEVGKVKKPFTPPREVHVQVTHSLAPEKREIFNSL |
| | NNWAQENILVLLKDVDKCWQPSDFLPDSASEGFDE |
| | QVMELRKRCKEIPDDYFIVLVGDMITEEALPTYQT |
| | MLNTLDGVRDETGASLTPWAIWTRAWTAEENRHGD |
| | LLNKYLYLSGRVDMKQIEKTIQYLIGSGMDPRTEN |
| | NPYLGFIYTSFQERATFISHGNTARLAKEHGDLKL |
| | AQICGIIAADEKRHETAYTKIVEKLFEIDPDGTVL |
| | ALADMMRKKVSMPAHLMYDGQDDNLFENFSSVAQR |
| | LGVYTAKDYADILEFLVGRWDIEKLTGLSGEGRKA |
| | QDYVCTLPPRIRRLEERAQSRVKKASATPFSWIFG |
| | REINLVD |
| 47 | MSFVKDFKPQALGDTNLFKPIKIGNNELLHRAVIP |
| | PLTRMRALHPGNIPNRDWAVEYYTQRAQRPGTMII |
| | TEGAFISPQAGGYDNAPGVWSEEQMVEWTKIFNAI |
| | HEKKSFVWVQLWVLGWAAFPDNLARDGLRYDSASD |
| | NVFMDAEQEAKAKKANNPQHSLTKDEIKQYIKEYV |
| | QAAKNSIAAGADGVEIHSANGYLLNQFLDPHSNTR |
| | TDEYGGSIENRARFTLEVVDALVEAIGHEKVGLRL |
| | SPYGVFNSMSGGAETGIVAQYAYVAGELEKRAKAG |
| | KRLAFVHLVEPRVTNPFLTEGEGEYEGGSNDFVYS |
| | IWKGPVIRAGNFALHPEVVREEVKDKRTLIGYGRF |
| | FISNPDLVDRLEKGLPLNKYDRDTFYQMSAHGYID |
| | YPTYEEALKLGWDKK |
| 48 | MASMVAFRPEAFLCFSPPKTTRSTRSPRISMASTV |
| | GPSTKVEIPKKPFMPPREVHVQVTHSMPPQKIEIF |
| | KSLEDWAENNILVHLKPVEKCWQPQDFLPDPSSEG |
| | FHEEVKELRERSKEIPDDYYVCLVGDMITEEALPT |
| | YQTMLNTLDGVRDETGASLTSWAVWTRAWTAEENR |
| | HGDLLNKYLYLSGRVDMKQIEKTIQYLIGSGMDPR |
| | TENSPYLGFIYTSFQERATFISHGNTARHAKEHGD |
| | VKLAQICGTIASDEKRHETAYTKIVEKLFEIDPDG |
| | TVLSFADMMKKKISMPAHLMYDGQDDNLFEHFSAV |
| | AQRLGVYTAKDYADILEFLINRWKVGELTGFSGEG |
| | KRAQDFVCTLAPRIRRIEERAQERAKQAPRIPFSW |
| | IYGREVQLVD |
| 49 | MEQPQAMEKVESVDVTEANAVAAGTNKLTKRIVAA |
| | GLGGKLMGTKSMVDVTADQLAKDSINELLARDKAL |
| | KEKYEKQKHISEQPWTKDNWHQHINWLNMILVCGL |
| | PLFGAIATYYKPPTKETVILGVVLYILGGLSITAG |
| | YHRLWSHRAYSARTPLKVLFALFGAGAVEGSIKWW |
| | SHSHRVHHRYTDTHRDPYDARKGFWYSHMGWMLTK |
| | PNPRYKARADISDLADDWIVRFQHRHYLLIMTFMA |
| | LILPTIIAKYFWDDAWGGFIYGGIFKVFVIQQATF |
| | CVNSLAHWIGVQPFDDRRTPRDHFLTAIVTFGEGY |
| | HNFHHEFPSDYRNALKWYQYDPTKIVIYLSSKVGL |
| | SYNLKTFSDNAIKQGLVQQQQKKLDSMRAHLNWGT |
| | PLQDLPVWDKSEFFEKSKDKKGLVIISGIVHNVEN |
| | FIGEHPGGEKLVKGALGKDATTAFNGGVYAHSNAA |
| | HNLLATMRVAVIRDGNANANTFSLQNEFLDKKAAA |
| | AAASN |
| 50 | MDIPTAKYSEEDMKAALQPTEPEFFKVKVSKKKKN |
| | RRKVKHDTTASTIRRHISESKWTISNWYRHIHWKN |
| | VVLVVIVPLLALLGSLTVPLTRTGFYWMGFNYIIT |
| | CTFVIMGYHRYWAHRSFQTTKEMSFLFAMVGASAG |
| | VGSAKWWCASHRAHHRFCDTERDPHNIRKGFWYSH |
| | FGWMLLIHHPKVQAAIKESESEDISSDPVILWQYE |
| | NYFQMFLVTGILIPAIIGWWLFQDFAGGLVYGGLV |
| | KIFLVQNTIFSVNSLCHCCGSQPFDDAKSSRNSIL |
| | LSLITFGEGQHNFHHEFPSDCRNGVEWYDFDPVKW |
| | VIFTLNALGVIYNVHAAPTTAINQLRVQQAQRILD |
| | KERSQLNWGIRIDKLPTLTSAEFSKLAKESQPQRA |
| | LVVISGIVHDVTPFLHDHPGGVALIKSSIGKDATN |
| | AFNGAVYSHSNAARNLLATMRIAVILGSEQEVWKQ |
| | QQKENKDVPLKNDSEGKKIVRSGEQITMTKTPSTT |
| | AGAA |
| 51 | MSAVTVTGNNGDASRSNTTTTTKRTGNVSSFSQSK |
| | GLTAIDTWGNVFKVPDFTIKQILDAIPKHCYERRL |
| | TTSFYYVFRDIFLIGCTMFMGSFIPMIENVFLRGA |
| | AYAALVFLLSVEYTGLWVLAHECGHQAFSDYGWVN |
| | DTVGWILHSYLLVPYFSWKYSHGKHHKATGHLTRD |
| | MVFVPATKEKFLEKRNASKLGELGEDAPIFTLYQL |
| | VAQQLGGWILYLFTNVTGQPYPNTPKWMQNHFVPS |
| | SPIFEKKDYWFIILSDLGILAQLMVLYVWRQQMGN |
| | WNLFIYWFLPYVLTNHWLVFITFLQHSDPTMPHYE |
| | AEQWTFARGAAATIDREFGFIGPFFFHDIIETHVL |
| | HHYVSRIPFYNAREASEGIKKVMGEHYRYSGENMW |
| | VSLWKSGRSCQFVDGENGVKMYRNINNWGIGTGEK |
| 52 | MSKVTVSGSEILEGSTKTVRRSGNVASFKQQKTAI |
| | DTFGNVFKVPDYTIKDILDAIPKHCYERSLVKSMS |
| | YVVRDIVAISAIAYVGLTYIPLLPNEFLRFAAWSA |
| | YVFSISCFGFGIWILGHECGHSAFSNYGWVNDTVG |
| | WVLHSLVMVPYFSWKFSHAKHHKATGHMTRDMVFV |
| | PYTAEEFKEKHQVTSLHDIAEETPIYSVFALLFQQ |
| | LGGLSLYLATNATGQPYPGVSKFFRSHYWPSSPVF |
| | DKKDYWYIVLSDLGILATLTSVYTAYKVFGFWPTF |
| | ITWFCPWILVNHWLVFVTFLQHTDSSMPHYDAQEW |
| | TFAKGAAATIDREFGILGIIFHDIIETHVLHHYVS |
| | RIPFYHAREATECIKKVMGEHYRHTDENMWVSLWK |
| | TWRSCQFVENHDGVYMFRNCNNVGVKPKDT |

In some embodiments, the enzyme catalyst includes an enzyme having an amino acid sequence as set forth in any one of SEQ ID NOs: 1-52, or any amino acid sequence having a sequence identity to any one of SEQ ID NOs: 1-52 of at least 75%, such as 75%, 80%, 85%, 90%, 91%, 92%, 93%, 94%, 95%, 96%, 97%, 98%, or 99% sequence homology to any one of SEQ ID NOs: 1-52, or a sequence homology within a range defined by any two of the aforementioned values.

In some embodiments, the enzyme catalysts are used in a soluble suspension or are immobilized to a solid substrate. In some embodiments the enzyme catalysts are immobilized to suitable polymeric supports by adsorption, affinity interactions, or covalent bonding. In some embodiments, the enzyme catalysts are self-immobilized through the formation of cross-linked aggregates, or entrapped into insoluble polymer microbeads. All of these immobilization approaches can be performed either by the natural physicochemical properties of the enzyme catalysts, or by addition of specific protein tags that allow these immobilization processes.

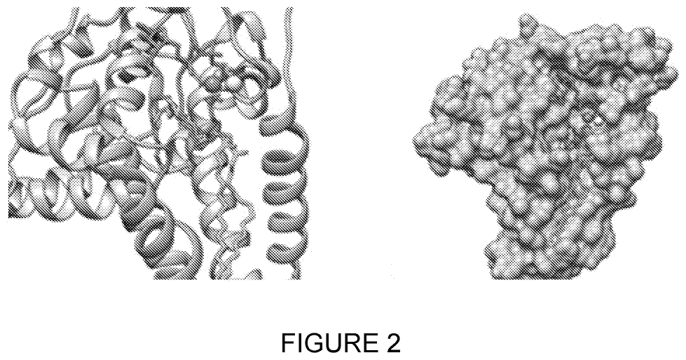

In some embodiments, the enzyme catalyst is an engineered desaturase enzyme. In some embodiments, the enzyme catalyst is a modified protein of the fatty acid desaturase group of enzymes that can be engineered to interact with a wider range of lipid molecules including mono-, di-, or triglycerides (as shown in FIG. 2). In some embodiments, the enzyme catalyst performs hydrogenase activity by metal substitution for transition metals, such as nickel, iron, palladium, or other transition metal ions.

In some embodiments, metal substitution can be performed by various approaches, including for example: removal of the native metal by a metal binding agent and/or competition with hydrogen ions at low pH, followed by insertion of the new metal; direct competition of the first metal by a different metal; and/or biosynthesis of the protein under enriched conditions of the metal of choice, such as nickel, iron, palladium, or other transition metal.

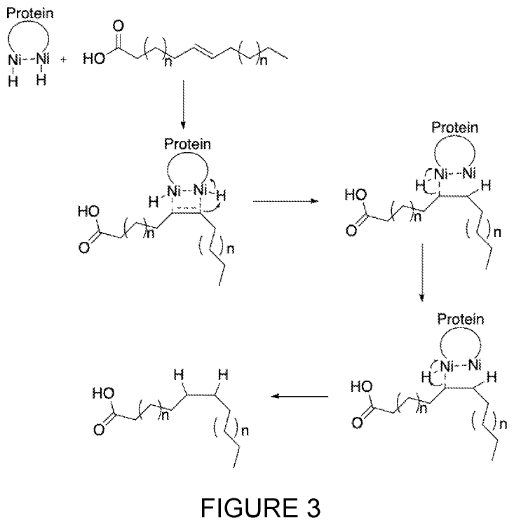

In some embodiments, oxidation of molecular hydrogen (H₂) and metal substitution can occur based on the mechanism shown in FIG. 3. Without wishing to be bound by theory, the mechanism depicted may be based on the molecular hydrogen oxidation by hydrogenases, in which dissociated protons have the potential to form hydride ions with the ion metals located inside the protein core, later promoting the oxidation and consequent reduction of a double carbon bond. The metal centers of the proteins are stabilized by residues such as histidines and cysteines, and the affinity of the protein toward metal ions can be improved by point and sequential amino acid substitutions of proximal residues to the metal core, by residues with high affinity toward transition metals, such as cysteine, histidine, aspartic acid, and/or glutamic acid residues.

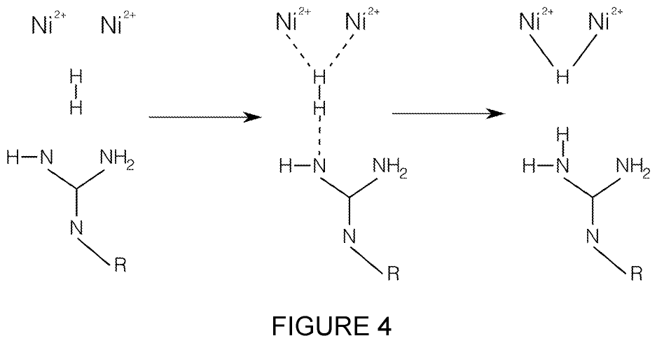

In some embodiments, the methods provided herein may further include the addition of a base, including a strong base. In some embodiments, addition of a base, such as arginine (R), to the active site can help in the reaction. Without wishing to be bound by theory, embodiments that include addition of a base may result in formation of a Frustrated Lewis pair reaction, which can be developed on site (FIG. 4), where the two metal ions (that act as a Lewis acid) may work in conjunction with the arginine (that works as the Lewis base) to split H₂ in 2H+.

The concept of Lewis acidity and basicity and the formation of simple Lewis acid-base adducts is a primary axiom of main group chemistry. (Lewis, Valence and the Structure of Atoms and Molecules, Chemical Catalogue Company, Inc., New York, 1923). The combination of Lewis donors and acceptors in which steric demands preclude formation of simple acid-base adducts have been termed “frustrated Lewis pairs” (FLPs) (Stephan, Org. Biomol. Chem., 2008, 6, 1535-1539).

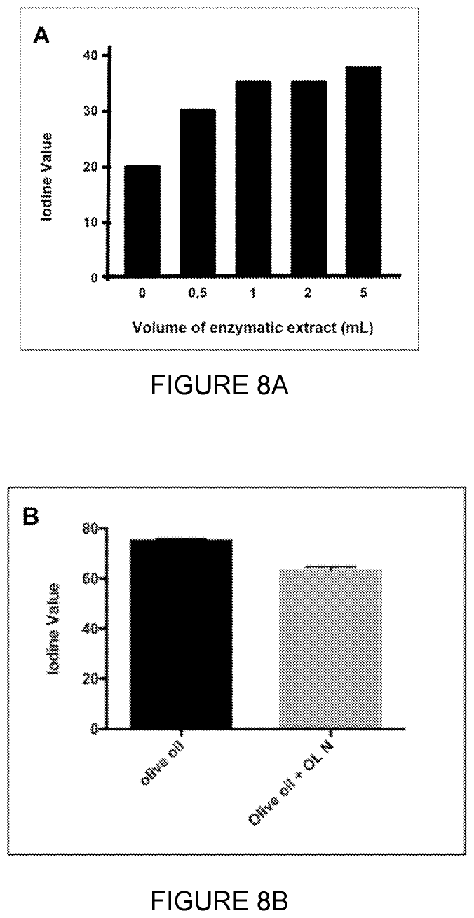

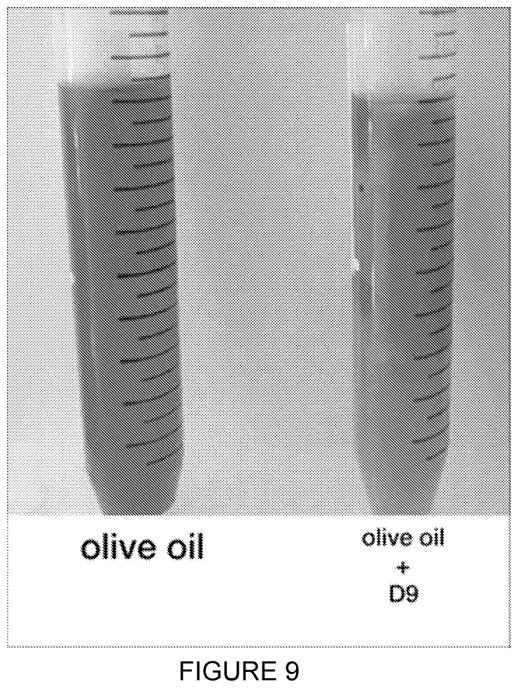

Results from the biochemical saturation process with the engineered desaturase enzymes are shown in FIGS. 8A and 8B, and FIG. 9. As used herein, the term “canonical” has its ordinary meaning as understood in light of the specification, and refers to a type that is typically found in a class. For example, a canonical substrate is a substrate that is typically found among substrate. Conversely, a non-canonical substrate is one that differs from canonical substrate, such as one that is not typical of the type of substrates.

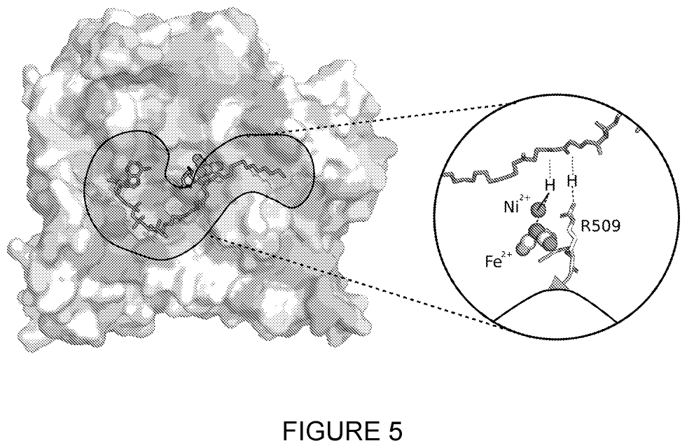

In some embodiments, the enzyme catalyst is an engineered hydrogenase enzyme. In some embodiments, the enzyme catalyst may be a modified hydrogenase to perform hydration of lipids by engineering a lipid binding region next to the metal core region (FIG. 5).

In some embodiments, the enzyme catalyst is a novel enzyme. In some embodiments, the enzyme catalyst may be a novel designed enzyme, containing a hydrogenase active site and a lipid-binding site.

In some embodiments, the hydrogenase active site includes a [Ni—Fe], [Fe—Fe], or [Ni—Ni] metal core, and an opposite arginine. In some embodiments, the metal binding site includes transition metals, such as nickel, iron, palladium, cobalt, scandium, vanadium, chromium, manganese, molybdenum, rhodium, or other transition metal.

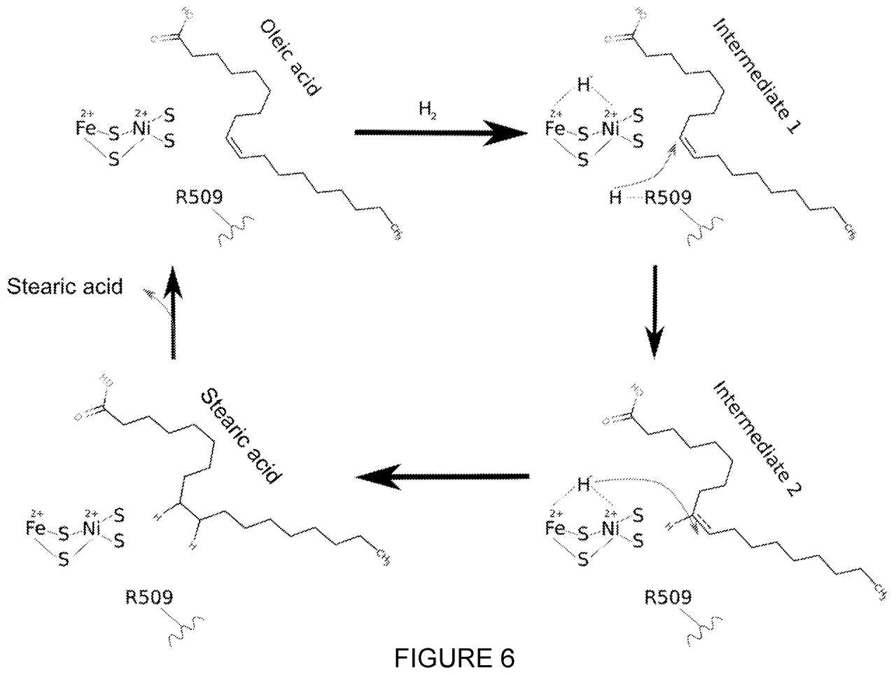

In some embodiments, the metal atoms bonded to the active site can capture molecular hydrogen gas molecules, orienting one of the hydrogen atoms towards the positively charged amino group of the arginine residue, producing a frustrated Lewis pair reaction splitting the hydrogen molecule into two hydrogen atoms. These hydrogen atoms are then transferred to the carbon-carbon double bond of the near lipid bonded to the lipid-binding active site, releasing a saturated lipid (FIG. 6).

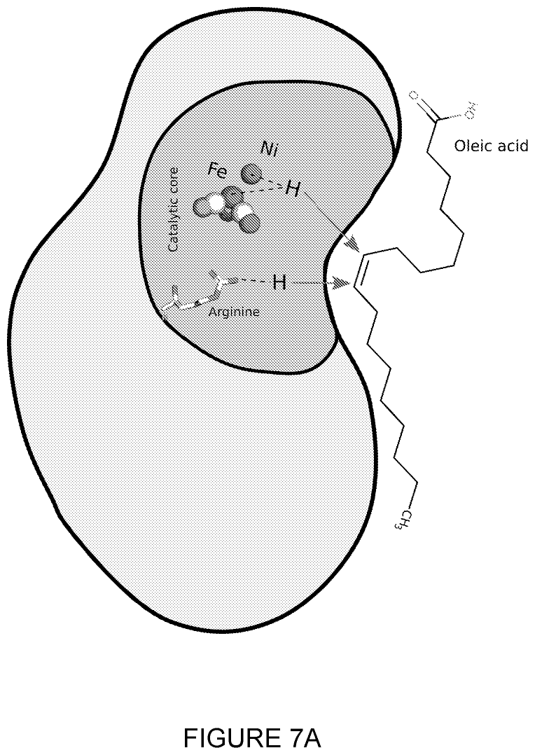

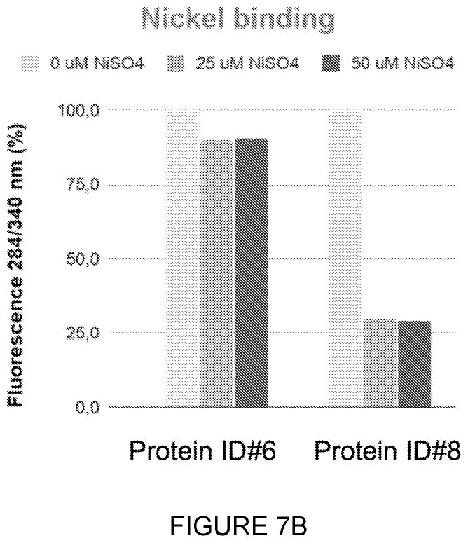

The lipid binding pocket can be designed in order to position the target carbon bond next to the hydrogenase site (FIG. 7A), and in this way different novel enzymes can be designed in order to saturate specific carbon atoms in the carbon chain.

In some embodiments, the enzyme catalyst is an engineered reductase enzyme. In some embodiments, the enzyme catalyst may be a lipid reductase enzyme, which normally reduces free fatty acids using redox cofactors (NADH, NADPH, FADH₂), that can be engineered to interact with a wider range of lipid molecules including mono-, di- and triglycerides. FIG. 7A depicts a representation of a protein, such as any of the proteins described in Table 1. The protein includes an active site, which includes a catalytic core, and an amino acid opposite the catalytic core. In some embodiments, the catalytic core is a metal core. In some embodiments, the catalytic core includes a nickel and an iron atom (Ni—Fe core). In some embodiments, the opposite amino acid is an arginine. In some embodiments, the active site is configured to bind a substrate, such as a lipid. In some embodiments, the lipid is oleic acid. As shown in FIG. 7B, nickel binding in a designed pocket greatly reduces the intrinsic protein fluorescence in the novel enzyme design (protein 3-SEQ ID NO: 3), but not to the same degree in a design lacking the nickel binding domain (protein 1-SEQ ID NO: 1).

In some embodiments, the methods include a post-hydrogenation process. In some embodiments, the product obtained from any of the hydrogenation reactions described herein are processed through a variety of methods in order to remove unwanted elements remnant from the enzymatic reaction, such as salts, leached metal particles, water, and water soluble components. These processing methods may include, for example, organic solvent extraction, sedimentation, filtration, vacuum treatment, centrifugation, and/or particle adsorption. These processes allow to recover all the fatty compounds, leaving the oil free of aqueous-phase or water-soluble components. In some embodiments, the methods allow recovery of target compounds. In some embodiments, unwanted organic solvents can also be removed from the processed product by rotavapor, distillation, vacuum treatment, or a combination thereof.

Additionally, the product obtained from the enzymatic saturation of oils can be further enriched in saturated lipids by dry fractionation, solvent-based fractionation, detergent-assisted fractionation, distillation, supercritical extraction or a combination of these methods. The product of enzymatic saturation of oils can be used as well in interesterification and oil-blending processes to further fine tune the physical properties of the product.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1: Production of Partially Hydrogenated Fatty Acid

The following example demonstrates an example method for producing a partially hydrogenated fatty acid.

An unsaturated starting material was obtained. Hydrogen gas was introduced into a reaction chamber, together with an enzyme catalyst (one or more of a hydrogenase enzyme, including, for example, any enzyme having an amino acid sequence as set forth in SEQ ID NOs: 1-52, or an enzyme having a sequence that is at least 75% identical to any one of SEQ ID NOs: 1-52, such as 75%, 80%, 85%, 90%, 91%, 92%, 93%, 94%, 95%, 96%, 97%, 98%, or 99% sequence identity to any one of SEQ ID NOs: 1-52). The starting material was added to the reaction chamber together with the hydrogen gas and the enzyme catalyst for a period of time sufficient to catalyze the starting material to at least partially hydrogenated products (partially saturated product).

In some embodiments, the enzyme catalyst was an engineered enzyme catalyst such as an enzyme catalyst as provided herein. In some embodiments, the enzyme catalyst included a metal core, such as a nickel-iron [Ni—Fe] core, and an opposite arginine. In some embodiments, the enzyme catalyst has an amino acid sequence as set forth in SEQ ID NO: 3.

The starting material was any suitable starting material, including, for example, any partially of fully unsaturated molecule, such as myristoleic acid, palmitoleic acid, sapienic acid, oleic acid, elaidic acid, vaccenic acid, linoleic acid, linoelaidic acid, alpha-linolenic acid, arachidonic acid, erucic acid, docosahexaenoic acid, and eicosapentaenoic acid. The saturated product may be any equivalent saturated product, including, for example, butyric acid, lauric acid, myristic acid, palmitic acid, or stearic acid.

### Example 2: Use of Desaturase Enzymes to Saturate Vegetable Oils

The coding sequence for the Olea europaea oleate desaturase enzyme (OLΔ9) was optimized for recombinant production in Pichia pastoris, and the obtained synthetic DNA was cloned downstream a methanol-inducible promoter for the recombinant production of the enzyme by secretion to the extracellular media. The production of OLΔ9 was monitored by concentrating the clarified culture supernatants by ammonium sulfate precipitation, followed by SDS-PAGE electrophoresis.

In order to detect the canonical desaturase enzymatic activity in the obtained precipitates, these were resolubilized by dialysis against phosphate buffer saline solution, and then mixed together with 0.05% Tween 20, 5 mM DTT and 1% stearic acid, and incubated at 30° C. for 24 h. After this time, fatty acids were extracted with ethyl acetate, and the saturation status assessed by iodine value. As shown in FIG. 8A, increasing concentrations of enzyme preparations generated the desaturation of stearic acid, reflected in the increase in iodine value.

The non-canonical saturation with the OLΔ9 enzyme was tested by recreating the conditions described above, but in a vessel saturated with hydrogen gas. Stearic acid was replaced with 90% olive oil, and the reaction was performed for a time period of 5 hours. As shown in FIG. 8B, iodine value of the extracted oil showed a decrease in saturation level. Additionally, two phases of the oil were obtained, with an upper fraction showing a solid, higher melting point behavior (FIG. 9)

### Example 3: Use of Engineered Hydrogenase Enzymes to Saturate Free Fatty Acids and Vegetable Oils

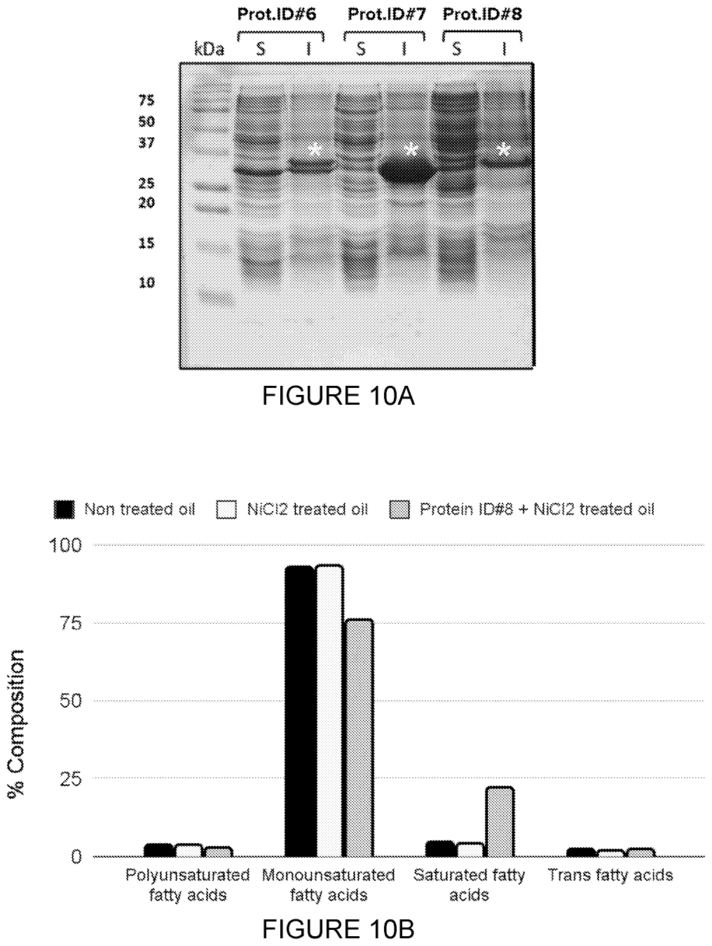

The coding sequence for engineered hydrogenase enzymes Protein-ID #6 (SEQ ID NO: 6), Protein-ID #7 (SEQ ID NO: 7), and Protein-ID #8 (SEQ ID NO: 8) were optimized for recombinant production in Escherichia coli, fused to 6×HIS sequence for immobilized metal affinity chromatography (IMAC), and the obtained synthetic DNA was cloned downstream an IPTG-inducible promoter for the recombinant production of the enzyme. Enzymes of 35 kDa were readily detected in the insoluble (I) fractions of cell lysates (FIG. 10A), confirming recombinant expression.

Protein-ID #8 (SEQ ID NO: 8) was chosen for saturation of oleic acid. To this end, after induction of recombinant Protein-ID #8 (SEQ ID NO: 8) expression, E. coli cells were lysed by sonication, and the insoluble protein fraction recovered by centrifugation. Proteins were solubilized in a buffer that included 8 M urea, 0.1% Triton X-100, 5 mM DTT, 100 mM Tris buffer and Protein-ID #8 (SEQ ID NO: 8) purified by IMAC. After elution, the obtained proteins were subjected to dialysis in refolding buffer (20 mM HEPES, 300 mM NaCl, 0.5 M trehalose, at a pH of 7.5), with a stepwise decrease in urea concentration to allow renaturation of Protein-ID #8 (SEQ ID NO: 8). Finally, the protein was lyophilized and later used in hydrogenation reactions. Briefly, proteins were mixed with 0.01 g of NiCl₂·6 H₂O, 0.1 g of oleic acid, 30 mL of chloroform, and 10 mL of water in a boiling flask, and incubated at 40° C. for a time period of 8 hours, in an atmosphere saturated with H₂ gas. After this period, the reaction mixture was cooled to room temperature and the volatiles were evaporated under vacuum. As a control, the same reaction was run with all components except Protein-ID #8 (SEQ ID NO: 8). The crude products were filtered through celite using ethyl acetate and the volatiles were evaporated under vacuum. The obtained samples were analyzed by GC-FID analysis, and fatty acid composition compared to an unsaturated oleic acid sample (FIG. 10B).

The obtained results surprisingly showed that when NiCl₂ was used alone for the saturation reaction, no significant changes were observed compared to the unsaturated oleic acid control. In contrast, when Protein-ID #8 (SEQ ID NO: 8)+NiCl₂ was used as a catalyst, a significant decrease in oleic acid, and concomitant increase in stearic acid was observed. No changes in trans fatty acids were observed.

### Example 4: Use of Metal Binding Enzymes to Saturate Vegetable Oils

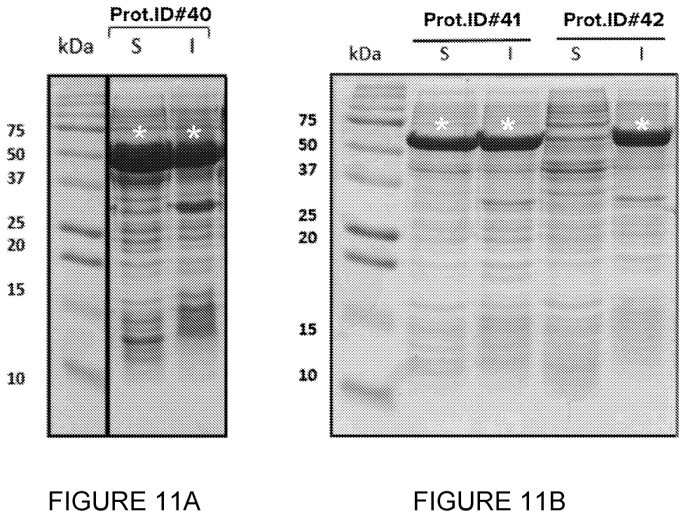

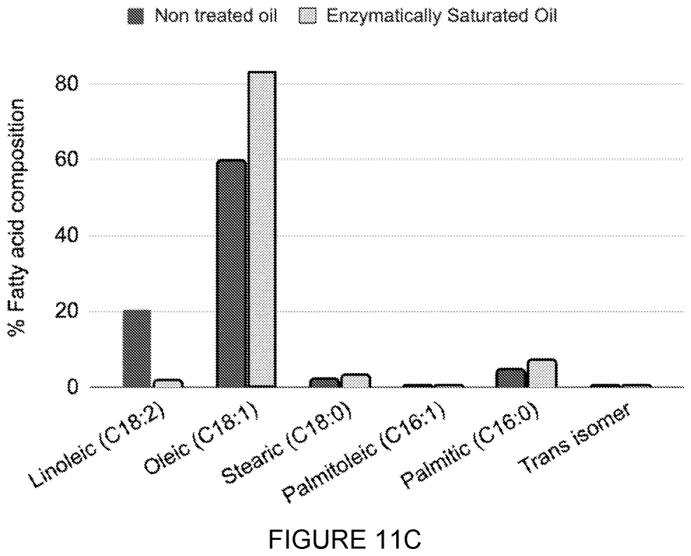

The coding sequence for Protein-ID #40 (SEQ ID NO: 40) was optimized for recombinant production in Escherichia coli, and the obtained synthetic DNA was cloned downstream an IPTG-inducible promoter for the recombinant production of the enzyme. An enzyme of 53 kDa was readily detected in the soluble (S) and insoluble (I) fractions of cell lysates (FIG. 11A), confirming good recombinant expression.

A second generation of engineered enzymes was also developed in order to generate variants adding one additional nickel binding site (Protein-ID #41-SEQ ID NO: 41) and also improving the affinity towards free fatty acids (Protein-ID #42-SEQ ID NO: 42). When these were expressed in a recombinant manner as described above, good protein expression was also demonstrated (FIG. 11B).

The ability of Protein-ID #40 (SEQ ID NO: 40) to be used to saturate canola oil was tested. To this end, after induction of expression of recombinant Protein-ID #40 (SEQ ID NO: 40) in E. coli, cells were lysed by sonication, centrifuged to remove insoluble material, and metal binding proteins of interest purified by immobilized metal affinity chromatography. After elution, the obtained proteins were subjected to dialysis against HEPES buffer to remove unwanted salts and metals, and lyophilized. The obtained proteins were then used in hydrogenation reactions. Briefly, proteins were mixed with 0.01 g of NiCl₂·6 H₂O, 0.1 g of canola oil, 30 mL of chloroform, and 10 mL of water in a boiling flask, and incubated at 40° C. for 8 hours, in an atmosphere saturated with H₂ gas. After this period, the reaction mixture was cooled to room temperature and the volatiles were evaporated under vacuum. The crude product was filtered through celite using ethyl acetate and the volatiles were evaporated under vacuum. The obtained sample was analyzed by GC-FID analysis, and fatty acid composition compared to an unsaturated canola oil sample (FIG. 11C).

The obtained results unexpectedly showed that the reaction allows the partial hydrogenation of canola oil, significantly decreasing the concentration of linoleic acid (C18:2), increasing oleic acid concentration (C18:1), and avoiding any increase in trans fatty acids. These results show that this method can generate stereospecific catalytic hydrogenation of vegetable oils, and avoid the generation of troublesome trans fatty acids.

### Example 5: Use of Novel Enzyme to Partially Saturate Canola Oil

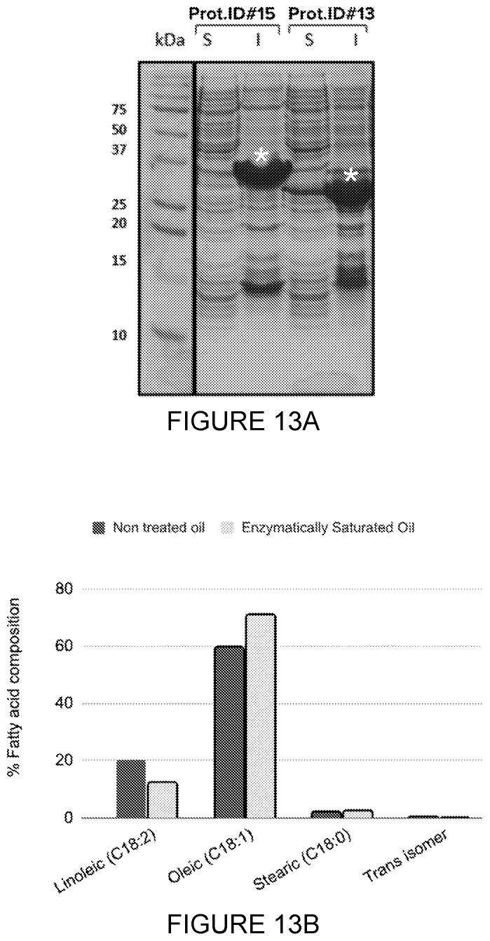

The protein sequence for natural Protein-ID #13 (SEQ ID NO: 13) was modified to contain a metal binding domain, and further modified in order to increase its affinity towards free fatty acids, generating novel enzyme Protein-ID #15 (SEQ ID NO: 15). The coding sequences for these proteins were optimized for expression in Escherichia coli, and the obtained synthetic DNA cloned downstream an IPTG-inducible promoter, and upstream of a in frame 6×HIS tag for the recombinant production of the enzyme. Protein-ID #13 (SEQ ID NO: 13) and #15 (SEQ ID NO: 15) of 31 and 32 kDa respectively could be readily detected in the insoluble (I) fractions of cell lysates (FIG. 13A), confirming good recombinant expression, although Protein-ID #13 (SEQ ID NO: 13) showed a size slightly smaller than expected.

The ability of Protein-ID #15 (SEQ ID NO: 15) to be used to partially saturate canola oil was put to a test. To this end, after induction of expression of recombinant Protein-ID #15 in E. coli, cells were lysed by sonication, and the insoluble protein fraction recovered by centrifugation. Proteins were solubilized in an 8 M urea, 0.1% Triton X-100, 5 mM DTT, 100 mM Tris buffer and Protein-ID #15 (SEQ ID NO: 15) purified by IMAC. After elution, the obtained proteins were subjected to dialysis in refolding buffer (20 mM HEPES, 300 mM NaCl, 0.5 M Trehalose, pH 7.5), with a stepwise decrease in Urea concentration to allow protein renaturation. Finally, the protein was lyophilized and later used in hydrogenation reactions: 68 mg of pure Protein-ID #15 (SEQ ID NO: 15) was mixed with 0.01 g of NiCl₂·6 H₂O, 0.1 g of canola oil, 15 mL of chloroform and 6 mL of water in a boiling flask, and incubated at 40° C. for 8 hours, in an atmosphere saturated with H₂ gas. After this period, the reaction mixture was cooled to room temperature and the volatiles were evaporated under vacuum. The crude products were filtered through celite using ethyl acetate and the volatiles were evaporated under vacuum. The obtained samples were analyzed by GC-FID analysis, and fatty acid composition compared to an unsaturated oleic acid sample (FIG. 13B).

The obtained results show that the reaction allows the partial hydrogenation of canola oil, decreasing the concentration of linoleic acid (C18:2), increasing oleic acid concentration (C18:1) and avoiding any increase in trans fatty acids. These results show that this Protein and method can generate stereospecific catalytic hydrogenation of vegetable oils, and avoid the generation of troublesome trans fatty acids.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through second aspects is applicable to all aspects and embodiments identified herein. Moreover, any of the features of an embodiment of the first through third aspects is independently combinable, partly or wholly with other embodiments described herein in any way, e.g., one, two, or three or more embodiments may be combinable in whole or in part. Further, any of the features of an embodiment of the first through third aspects may be made optional to other aspects or embodiments.

What is claimed is:

1. A method of saturating an unsaturated molecule, the method comprising:
contacting an unsaturated molecule with an enzyme to produce a saturated molecule; and
recovering the saturated molecule.

2. The method of claim 1, wherein the saturated molecule is fully or partially saturated.

3. The method of claim 1, wherein the unsaturated molecule comprises an unsaturated alkene.

4. The method of claim 1, wherein the unsaturated molecule is an unsaturated triglyceride or a free fatty acid.

5. The method of claim 1, wherein the unsaturated molecule is vegetable oil.

6. The method of claim 1, wherein the unsaturated molecule is olive oil or canola oil.

7. The method of claim 1, wherein contacting the unsaturated molecule with the enzyme is performed in a solvent.

8. The method of claim 1, wherein the contacting is performed for a sufficient period of time to allow at least partial saturation.

9. The method of claim 1, wherein the enzyme is in solution, or wherein the enzyme is immobilized.

10. The method of claim 9, wherein the enzyme is immobilized on a polymeric support.

11. The method of claim 10, wherein the polymeric support is an insoluble polymer microbead.

12. The method of claim 1, wherein the enzyme is prepared by protein fermentation or chemical synthesis.

13. The method of claim 1, wherein the enzyme is a purified enzyme.

14. The method of claim 1, wherein the enzyme is a recombinant nickel binding enzyme.

15. The method of claim 1, wherein the enzyme comprises a consensus sequence as set forth in SEQ ID NO: 53.

16. The method of claim 1, wherein the enzyme has an amino acid sequence as set forth in SEQ ID NOs: 1-52, or having a sequence identity of at least 75% to any one of SEQ ID NOs: 1-52.

17. The method of claim 1, wherein the enzyme has an amino acid sequence as set forth in SEQ ID NO: 15 or 40, or having a sequence identity of at least 75% to any one of SEQ ID NOs: 15 or 40.

18. The method of claim 1, wherein the enzyme is a novel designed protein having hydrogenase activity and comprising a substrate specific binding site.

19. The method of claim 18, wherein said substrate specific binding site comprises one or more alkene unsaturation sites.

20. The method of claim 1, wherein said enzyme is a hydrogenase enzyme engineered to bind a non-canonical substrate.

21. The method of claim 20, wherein said non-canonical substrate comprises one or more alkene unsaturation sites.

22. The method of claim 20, wherein the hydrogenase enzyme comprises a modified hydrophobic portion that supports the recognition of an unsaturated acyl chain.

23. The method of claim 1, wherein said enzyme is a dehydrogenase enzyme engineered to bind a non-canonical substrate.

24. The method of claim 23, wherein said non-canonical substrate comprises one or more alkene unsaturation sites.

25. The method of claim 1, wherein said enzyme is a desaturase enzyme.

26. The method of claim 25, wherein said desaturase enzyme is engineered.

27. The method of claim 26, wherein said desaturase enzyme is engineered to bind a transition metal in its active site.

28. The method of claim 27, wherein said active site comprises at least 2 cysteine residues that support transition metal binding.

29. The method of claim 27, wherein the transition metal is nickel, iron, or palladium.

30. The method of claim 27, wherein said active site comprises an arginine residue that is configured to support a frustrated Lewis pair reaction.


SEQUENCE LISTING (53 sequences)

SEQ ID NO: 1 - protein, 219 aa, synthetic construct
  REGION 1..219: note = Synthetic or recombinant peptide
VISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIER

SEQ ID NO: 2 - protein, 219 aa, synthetic construct
  REGION 1..219: note = Synthetic or recombinant peptide
VISYDNYVTILDEETLKAWIAKAEKAPVFAFDTHTDSLDNISANLVGLSFAIEPGVAAYIPVAHDYLDAPDQISRERALELLKPLLEDEKALKVGHELKHDRGILANYGIELRGIAFDTMLESYILNSVAGRHDMDSLAERWLKHKTITFEEIAGKGKNQLTFNQIALEEAGRHAAEHADVTLQLHLKMWPDLQKHKGPLNVFENIEMPLVPVLSRIER

SEQ ID NO: 3 - protein, 219 aa, synthetic construct
  REGION 1..219: note = Synthetic or recombinant peptide
VISYDNYVTILDEETLKAWIAKAEKAPVFAFDTCTCCCSNISALGCNVLCLIEPGVAAYIPVAHDYLDAPDQISRERALELLKPLLEDEKALKCHIELKLCRDGICNYGIELRGIAFCCIFESYILNSVAGRHCCCCLAERWLKHKTITFEEIAGKGKNQLTFCQIALEEAGRHACDCARHTLQLHLKMWPDLQKHKGPLNVFENIEMPLVPVLSRIER

SEQ ID NO: 4 - protein, 219 aa, synthetic construct
  REGION 1..219: note = Synthetic or recombinant peptide
VISYDNYVTILDEETLKAWIAKAEKAPVFAHDHHTDSLDNISANLVGLSFAIEPGVAAYIPVAHDYLDAPDQISRERALELLKPLLEDEKALKVGHELKHDRGILANYGIELRGIAFDTMLESYILNSVAGRHDMDSLAERWLKHKTITFEEIAGKGKNQLTFNQIALEEAGRHAAEHADVTLQLHLKMWPDLQKHKGPLNVFENIEMPLVPVLSRIER

SEQ ID NO: 5 - protein, 219 aa, synthetic construct
  REGION 1..219: note = Synthetic or recombinant peptide
VISYDNYVTILDEETLKAWIAKAEKAPVFADCHCTFEQNNISAMKCCQAICIEPGVAAYIPVAHDYLDAPDQISRERALELLKPLLEDEKALKHCCELPFFRCCCMNYGIELRGIAFYCFCESYILNSVAGRHAWRCLAERWLKHKTITFEEIAGKGKNQLTACQIALEEAGRHACCCAVYTLQLHLKMWPDLQKHKGPLNVFENIEMPLVPVLSRIER

SEQ ID NO: 6 - protein, 291 aa, synthetic construct
  REGION 1..291: note = Synthetic or recombinant peptide
AFWGASNELLHDPTMIKEGSSWYALGTGLTEERGLRVLKSSDAKNWTVQKSIFTTPLSWWSNYVPNYGQNQWAPDIQYYNGKYWLYYSVSSFGSNTSAIGLASSTSISSGGWKDEGLVIRSTSSNNYNAIDPELTFDKDGNPWLAFGSFWSGIKLTKLDKSTMKPTGSLYSIAARPNNGGALEAPTLTYQNGYYYLMVSFDKCCDGVNSTYKIAYGRSKSITGPYLDKSGKSMLEGGGTILDSGNDQWKGPGGQDIVNGNILVRHAYDANDNGIPKLLINDLNWSSGWPSY

SEQ ID NO: 7 - protein, 291 aa, synthetic construct
  REGION 1..291: note = Synthetic or recombinant peptide
AFWGASNELLHDPTMIKEGSSWYALGTGLTEERGLRVLKSSDAKNWTVQKSIFTTPLSWWSNYVPNYGQNQWRPDIQYYNGKYWLYYSVSSFGSNTSAIGLASSTSISSGGWKDEGLVIRSTSSNNYDAEDPELTFDKDGNPWLAFGDFRSGIKLTKLDKSTMKPTGSLYSIAARPNNGGALEAPTLTYQNGYYYLMVSFDKCCDGVNSTYKIAYGRSKSITGPYLDKSGKSMLEGGGTILDSGNDQWKGPGGQDIVNGNILVRHAYDANDNGIPKLLINDLNWSSGWPSY

SEQ ID NO: 8 - protein, 291 aa, synthetic construct
  REGION 1..291: note = Synthetic or recombinant peptide
AFWGASNELLHDPTMIKEGSSWYALMTGLTEERGLRVLKSSDAKNWTVQKSIFTTPLSWWSNYVPKYGQNQWRPDIQYYNGKYWLYYSVWSFGSNTSAIGLASSTSISSGGWKDEGLVIYSTSSNNYDAEDDELTFDKDGNPWLAFGDFRSGIKLTKLDKSTMKPTGSLYSIAMRPNNGGALEAPTLTYQNGYYYLMVSFFKCCWGVNFTYKIAYGRSKSITGPYLDKSGKSMLEGGGTILDSGNDQWKGPGGQDIVNGNILVRHAYDANDNGIPKLLINDLNWSSGWPSY

SEQ ID NO: 9 - protein, 291 aa, synthetic construct
  REGION 1..291: note = Synthetic or recombinant peptide
AFWGASNELLHDPTMIKEGSSWYALGTGLTEERGLRVLKSSDAKNWTVQKSIFTTPLSWWSNYVPNYGQNQHAPDIQYYNGKYWLYYSVESFGSNTSAIGLASSTSISSGGWKDEGLVIRSTSSNNYHADHPELTFDKDGNPWLAFGHHESGIKLTKLDKSTMKPTGSLYSIAARPNNGGALHAPTLTYQNGYYYLMVSFDKCCDGVNSTYKIAYGRSKSITGPYLDKSGKSMLEGGGTILDSGNDQWKGPGGQDIVNGNILVRHAYDANDNGIPKLLINDLNWSSGWPSY

SEQ ID NO: 10 - protein, 291 aa, synthetic construct
  REGION 1..291: note = Synthetic or recombinant peptide
AFWGASNELLHDPTMIKEGSSWYALGTGLTEERGLRVLKSSDAKNWTVQKSIFTTPLSWWSNYVPNYGQNQHAPDIQYYNGKYWLYYSVESCGCKCCVIGLASSTSISSGGWKDEGLVIRSTSSNNYLACHPELTFDKDGNPWLAFGCHESCCCLTKLDKSTMKPTGSLYSIAARPNNGGNSVACTLTYQNGYYYLMVSFDKCCDGVCCACKIAYGRSKSITGPYLDKSGKSMLEGGGTILDSGNDQWKGPGGQDIVNGNILVRHAYDANDNGIPKLLINDLNWSSGWPSY

SEQ ID NO: 11 - protein, 291 aa, synthetic construct
  REGION 1..291: note = Synthetic or recombinant peptide
AFWGASNELLHDPTMIKEGSSWYALGTGLTEERGLRVLKSSDAKNWTVQKSIFTTPLSWWSNYVPNYGQNQHAPDIQYYNGKYWLYYSVESFGSNTSAIGLASSTSISSGGWKDEGLVIRSTSSNNYHACHPELTFDKDGNPWLAFGCCESGIKLTKLDKSTMKPTGSLYSIAARPNNGGALDAPTLTYQNGYYYLMVSFEKCCDGVRSTYKIAYGRSKSITGPYLDKSGKSMLEGGGTILDSGNDQWKGPGGQDIVNGNILVRHAYDANDNGIPKLLINDLNWSSGWPSY

SEQ ID NO: 12 - protein, 291 aa, synthetic construct
  REGION 1..291: note = Synthetic or recombinant peptide
AFWGASNELLHDPTMIKEGSSWYALGTGLTEERGLRVLKSSDAKNWTVQKSIFTTPLSWWSNYVPNYGQNQHAPDIQYYNGKYWLYYSCRSYCCCSCCIGLASSTSISSGGWKDEGLVIRSTSSNNYHACHPELTFDKDGNPWLAFGCCESCCCLTKLDKSTMKPTGSLYSIAARPNNGCCCCACTLTYQNGYYYLMVSFEKCCDGVYSCCKIAYGRSKSITGPYLDKSGKSMLEGGGTILDSGNDQWKGPGGQDIVNGNILVRHAYDANDNGIPKLLINDLNWSSGWPSY

SEQ ID NO: 13 - protein, 254 aa, synthetic construct
  REGION 1..254: note = Synthetic or recombinant peptide
MNYLNNIRIENPLTICYTNDVVKNFTANGLLSIGASPAMSEAPEEAEEFYKVAQALLINIGTLTAQNEQDIIAIAQTANEAGLPIVFDPVAVGASTYRKQFCKLLLKSAKVSVIKGNASEILALIDDTATLDAVTIAKKAYAIYKTAIVITGKEDVIVQGDKAIVLANGSPLLARVTGAGCLLGGIIAGFLFRETEPDIEALIEAVSVFNIAAEVAAENENCGGPGTFSPLLLDTLYHLNETTYQQRIRIQEVE

SEQ ID NO: 14 - protein, 254 aa, synthetic construct
  REGION 1..254: note = Synthetic or recombinant peptide
MNYLNNIRIENPLTICYTNDVVKNFTANGLLSIGASPAMSEAPEEAEEFYKVAQALLINIGTLTAQNEQDIIAIAQTANEAGLPIVFDPVAVGASTYRKQFCKLLLKSAKVSVIKGNASEILALIDDTATLDAVTIAKKAYAIYKTAIVIHGKEDVIVQGDKAIVLANGSPLLARVTGAGCLLGGIIAGFLFRETEPDIEALIEAESVHHIAAEVAAENENCGGPGTFSPLLLDTLYHLNETTYQQRIRIQEVE

SEQ ID NO: 15 - protein, 254 aa, synthetic construct
  REGION 1..254: note = Synthetic or recombinant peptide
MNYLNNIRIENPLTICYTNDVVKNFTANGLLSIGASPAMSEAPEEAEEFYKVAQALLINIGTLTAQNEQDIIAIAQTANEAGLPIVFDPVAVGASTYRKQFCKLLLKSAKVSVIKCIYNEILALIDDTATLDAVTIAKKAYAIYKTAIVPMGKEVVSWQGDKAIHFDPPMPSHFPVTGAGCLGMTISAGFLFRETEPDIEALDTFCSCCHIYGCEAAENENCGGPGTFSPLLLDTLYHLNETTYQQRIRIQEVE

SEQ ID NO: 16 - protein, 254 aa, synthetic construct
  REGION 1..254: note = Synthetic or recombinant peptide
MNYLNNIRIENPLTICYTNDVVKNFTANGLLSIGASPAMSEAPEEAEEFYKVAQALLINIGTLTAQNEQDIIAIAQTANEAGLPIVFDPVAVGASTYRKQFCKLLLKSAKVSVIKGNASEILALIDDTATLDAVTIAKKAYAIYKTAIVIHGKEHVIVQGDKAIVLANGSPLEARVTGAGCLHGGIIAGFLFRETEPDIEALIEAHSVHHIAAEVAAENENCGGPGTFSPLLLDTLYHLNETTYQQRIRIQEVE

SEQ ID NO: 17 - protein, 254 aa, synthetic construct
  REGION 1..254: note = Synthetic or recombinant peptide
MNYLNNIRIENPLTICYTNDVVKNFTANGLLSIGASPAMSEAPEEAEEFYKVAQALLINIGTLTAQNEQDIIAIAQTANEAGLPIVFDPVAVGASTYRKQFCKLLLKSAKVSVIKAKDMEILALIDDTATLDAVTIAKKAYAIYKTAIVKYGCGCVSKQGDKAICCCEGRWKQARVTGCACDFGNLIAGFLFRETEPDIEALECCCSMWHIQCWEAAENENCGGPGTFSPLLLDTLYHLNETTYQQRIRIQEVE

SEQ ID NO: 18 - protein, 254 aa, synthetic construct
  REGION 1..254: note = Synthetic or recombinant peptide
MNYLNNIRIENPLTICYTNDVVKNFTANGLLSIGASPAMSEAPEEAEEFYKVAQALLINIGTLTAQNEQDIIAIAQTANEAGLPIVFDPVAVGASTYRKQFCKLLLKSAKVSVIKGNASEILALIDDTATLDAVTIAKKAYAIYKTAIVICGKECVIVQGDKAIVLANGSPLEARVTGAGCLCGGIIAGFLFRETEPDIEALIEAHSVHCIAAEVAAENENCGGPGTFSPLLLDTLYHLNETTYQQRIRIQEVE

SEQ ID NO: 19 - protein, 254 aa, synthetic construct
  REGION 1..254: note = Synthetic or recombinant peptide
MNYLNNIRIENPLTICYTNDVVKNFTANGLLSIGASPAMSEAPEEAEEFYKVAQALLINIGTLTAQNEQDIIAIAQTANEAGLPIVFDPVAVGASTYRKQFCKLLLKSAKVSVIKVQCPEILALIDDTATLDAVTIAKKAYAIYKTAIVICGWSCVHQQGDKAIIVCKGHMTAWKVTGLWCLCGSMVCGFLFRETEPDIEALEGCISHICICVKKAAENENCGGPGTFSPLLLDTLYHLNETTYQQRIRIQEVE

SEQ ID NO: 20 - protein, 254 aa, synthetic construct; identical to SEQ ID NO: 19
  REGION 1..254: note = Synthetic or recombinant peptide

SEQ ID NO: 21 - protein, 254 aa, synthetic construct
  REGION 1..254: note = Synthetic or recombinant peptide
MNYLNNIRIENPLTICYTNDVVKNFTANGLLSIGASPAMSEAPEEAEEFYKVAQALLINIGTLTAQNEQDIIAIAQTANEAGLPIVFDPVAVGASTYRKQFCKLLLKSAKVSVIKGNASEILALIDDTATLDAVTIAKKAYAIYKTAIVIIEEGDVIVQGDKAIVLANGSPLLARVTGAGCLHGGIIAGFLFRETEPDIEALIEACSVIEIAAEVAAENENCGGPGTFSPLLLDTLYHLNETTYQQRIRIQEVE

SEQ ID NO: 22 - protein, 348 aa, synthetic construct
  REGION 1..348: note = Synthetic or recombinant peptide
MSALTDLFPLPIVQAPMAGGVSVPQLAAAVCEAGGLGFLAAGYKTADGMYQEIKRLRGLTGRPFGVNVFMPQPELGAVEVYAHQLAGEAAWYETELGDPDGGRDDGYDAKLAVLLDDPVPVVSFHFGVPDREVIARLRRAGTLTLVTATTPEEARAVEAAGADAVIAQGVEAGGHQGTHRDSSEDDGAGIGLLSLLAQVREAVDIPVVAAGGIMRGGQIAAVLAAGADAAQLGTAFLATDESGAPGPHKRALTDPLFARTRLTRAFTGRPARSLVNRFLREHGPYAPAAYPDVHHLTSPLRKAAAKAGDAQGMALWAGQGHRMARELPAGRLVEVLAAELAEARTALS

SEQ ID NO: 23 - protein, 348 aa, synthetic construct
  REGION 1..348: note = Synthetic or recombinant peptide
MSALTDLFPLPIVQHDHHGGVSVPQLAAAVCEAGGLGFLAAGYKTADGMYQEIKRLRGLTGRPFGVDVHMPQPELGAVEVYAHQLAGEAAWYETELGDPDGGRDDGYDAKLAVLLDDPVPVVSFHFGVPDREVIARLRRAGTLTLVTATTPEEARAVEAAGADAVIAQGVEAGGHQGTHRDSSEDDGAGIGLLSLLAQVREAVDIPVVAAGGIMRGGQIAAVLAAGADAARLGTAFLATDESGAPGPHKRALTDPLFARTRLTRAFTGRPARSLVNRFLREHGPYAPAAYPDVHHLTSPLRKAAAKAGDAQGMALWAGQGHRMARELPAGRLVEVLAAELAEARTALS

SEQ ID NO: 24 - protein, 348 aa, synthetic construct
  REGION 1..348: note = Synthetic or recombinant peptide
MSALTDLFPLPIVQHDHHLGASVPQLAAAVCEAGGLGFLAARYKTADGMYQEIKRLRGLTGRFFGVDVHMPQPELGAVEVYAHQLAGEAAWYETELGDPDGGRDDGYDAKLAVLLDDPVPVVSFHFGVPDREVIARLRRAGTWTLFTATTPEEARAVEAAGADAVIAQGVTAGGHQGSHRDSSEDDGAGIGLLSLLAQVREAVDIPVVAAGGTMRGGQIAALLAAGFDAARLGTAFLATDESGAPGPHKRALTDPLFARTRLTRAFTGRPARSLVNRFLREHGPYAPAAYPDVHHLTQPLRKAWYKAGDAQGMALWAGQGHRMARELPAGRLVEVLAAELAEARTALS

SEQ ID NO: 25 - protein, 184 aa, synthetic construct
  REGION 1..184: note = Synthetic or recombinant peptide
MQTFVHEAGRLPAHIAAELGSYRYRVFVEQLGWQLPSEDEKMERDQYDRDDTVYVLGRDANGEICGCARLLPTTRPYLLQEVFPHLLADEAPRSAHVWELSRFAATAWSVRPMLAAAVECAARRGARQLIGVTFCSMERMFRRIGVHAHRAGAPVSIDGRMVVACWIDIDAQTLAALDLDPALC

SEQ ID NO: 26 - protein, 184 aa, synthetic construct
  REGION 1..184: note = Synthetic or recombinant peptide
MQTFVHEAGRLPAHIAAELGSYRYRVFVEQLGRQLPSEDEKMERDQYDRDDTVYVLGRDANGEICGCARLLPTTRPYLLQEVEPHELADEAPRSAHVWELSRFAATAWSVRPMLAAAVECAARRGARQLIGHTDCSMERMFRRIGVHAHRAGAPVSEDGRMEVACWIDIDAQTLAALDLDPALC

SEQ ID NO: 27 - protein, 184 aa, synthetic construct
  REGION 1..184: note = Synthetic or recombinant peptide
MQTFVHEAGRLPAHIAAELGSYRYQNFCSAQGRQLPSEDEKMERDQYDRDDTVYVLGRDANGEICGCYSRYPTTRPKDEVEFEPHEFADEAPRSAHVFPVSIFAWTAWSVRPMLAAAVECAARRGARQLIGDTECRMERMFRRIGVHAHRAGAGVVEEYRTEFICWIDIDAQTLAALDLDPALC

SEQ ID NO: 28 - protein, 526 aa, synthetic construct
  REGION 1..526: note = Synthetic or recombinant peptide
MQPDPIAADILRKEPEQETFVRLNVPLEVPTSDEVEAYKCLQECLELRKRYVFQETVAPWEKEEPFAHYPQGKSDHCFEMQDGVVHVFANKDAKEDLFPVADATAFFTDLHHVLKVIAAGNIRTLCHRRLVLLEQKFNLHLMLNADKEFLAQKSAPHRDFYNVRKVDTHVHHSACRFLGEITKQVFSDLEASKYQMAEYRISIYGRKMSEWDQLASWIVNNDLYSENVVWLIQLPRLYNIYKDMGIVTSFQNILDNIFIPLFEATVDPDSHPQLHVFLKQVVGFDLVDDESKPERRPTKHMPTPAQWTNAFNPAFSYYVYYCYANLYVLNKLRESKGMTTITLRPHSGEAGDIDHLAATFLTCHSIAHGINLRKSPVLQYLYYLAQIGLAMSPLSNNSLFLDYHRNPFPVFFLRGLNVSLSTDDPLQIHLTKEPLVEEYSIAASVWKLSACDLCEIARNSVYQSGFSHALKSHWIGKDYYKRGPDGNDIHKTNVPHIRVEFRDTIWKEEMQQVYLGKAVISDEVEH

SEQ ID NO: 29 - protein, 526 aa, synthetic construct
  REGION 1..526: note = Synthetic or recombinant peptide
MQPDPIAADILRKEPEQETFVRLNVPLEVPTSDEVEAYKCLQECLELRKRYVFQETVAPWEKEEPFAHYPQGKSDHCFEMQDGVVHVFANKDAKEDLFPVADATAFFTDLHHVLKVIAAGNIRTLCHRRLVLLEQKFNLHLMLNADKEFLAQKSAPHRDFYNVRKVDTHVHHSACRFLGEITKQVFSDLEASKYQMAEYRISIYGRKMSEWDQLASWIVNNDLYSENVVWLIQLPRLYNIYKDMGIVTSFQNILDNIFIPLFEATVDPDSHPQLHVFLKQVVGFDLVDDESKPERRPTKHMPTPAQWTNAFNPAFSYYVYYCYANLYVLNKLRESKGMTTITLRPHSGEAGDIDHLAATFLTCHSIAHGINLRKSPVLQYLYYLAEIGLAMSPLSNNSLFLDYHRNPFPVFFLRGLHVSLSTDDPLQIHLTKEPLVEEYSIAASVWKLSACDLCEIARESVHHSGFSHALKSHWIGKDYYKRGPDGNDIHKTNVPHIRVEFRDTIWKEEMQQVYLGKAVISDEVEH

SEQ ID NO: 30 - protein, 526 aa, synthetic construct
  REGION 1..526: note = Synthetic or recombinant peptide
MQPDPIAADILRKEPEQETFVRLNVPLEVPTSDEVEAYKCLQECAELMKRYVFQETVAPWEKEEPFAHYPQGKSDHCFEMQDGVVHVFANKDAKEDLFPVADATAFFTDLHHVLKVIAAGNIRTLCHRRLVLLEQKFNLHLMLNADKEFLAQKSAPHRDFYNVRKVDTHVHHSACRFLGEITKQVFSDLEASKYQMAEYRISIYGRKMSEWDQLASWIVNNDLYSENVVWLIQLPRLYNIYKDMGIVTSFQNILDNIFIPLFEATVDPDSHPQLHVFLKQVVGFDLVDDESKPERRPTKHMPTPAQWTNAFNPAFSYYVYYCYANLYVLNKLRESKGMTTITCRPHSGEAGDIDHLAATFLTCVSIAHGINLRKSPVLQYLYYLAEIFLAKSPLSNNSLFLDYHRNPFPVFFLRGLHVSCSTDDPLQIHLTKEPLVEEYSIAASVWKLSACDLCEIIRESVHHSGFSHALFSHWIGKDYYQRGPDGNDIHKTNVPHIRVEFRDTIWKEEMQQVYLGKAVISDEVEH

SEQ ID NO: 31 - protein, 273 aa, synthetic construct
  REGION 1..273: note = Synthetic or recombinant peptide
MLPNTGRLAGCTVFITGASRGIGKAIALKAAKDGANIVIAAKTAQPHPKLLGTIYTAAEEIEAVGGKALPCIVDVRDEQQISAAVEKAIKKFGGIDILVNNASAISLTNTLDTPTKRLDLMMNVNTRGTYLASKACIPYLKKSKVAHILNISPPLNLNPVWFKQHCAYTIAKYGMSMYVLGMAEEFKGEIAVNALWPKTAIHTAAMDMLGGIESQCRKVDIIADAAYSIFQKPKSFTGNFVIDENILKEEGIENFDVYAIKPGHPLQPDFFLD

SEQ ID NO: 32 - protein, 273 aa, synthetic construct
  REGION 1..273: note = Synthetic or recombinant peptide
MLPNTGRLAGCTVFITGASRGDGKAIALKAAKDGANIVIAAKTAQPHPKLLGTIYTAAEEIEAVGGKALPCIVDVRDEQQISAAVEKAIKKFGGIDILVNHAEAISLTNTLDTPTKRLDLMMNVNTRGTYLASKACIPYLKKSKVAHILNHSPPLNLNPVWFKQHCAYTIARYGMSMYVLGMAEEFKGEIAVNALWPKTAIHTAAMDMLGGIESQCRKVDIIADAAYSIFQKPKSFTGNFVIDENILKEEGIENFDVYAIKPGHPLQPDFFLD

SEQ ID NO: 33 - protein, 273 aa, synthetic construct
  REGION 1..273: note = Synthetic or recombinant peptide
MLPNTGRLAGCTVFITGASRGDGKNIAQKAAKDGANIVILAKTAQPHPKLLGTIYTAAEEIEAVGGKALPCIVDVRDEQQISAAVEKAIKKFGGIDILVNNAEAINLTNTLDTPTKRLDLMMWVNTRGPYLASKACIPYLKKSKVAHILNHSPPLNLNPVWFKQHCAYTIARYGMSMYVLGMAEEFKGEIAVNAHWPYTAIHTAAMDMCGGIESQCGKVDIIAWAAESIFQKPKSFTGNFVINENILKEEGIENFDVYAIKPGHPLQPDFFLD

SEQ ID NO: 34 - protein, 255 aa, synthetic construct
  REGION 1..255: note = Synthetic or recombinant peptide
MLSPAVQTFWKWLQEEGVITAKTPVKASVVTEGLGLVALKDISRNDVILQVPKRLWINPDAVAASEIGRVCSELKPWLSVILFLIRERSREDSVWKHYFGILPQETDSTIYWSEEELQELQGSQLLKTTVSVKEYVKNECLKLEQEIILPNKRLFPDPVTLDDFFWAFGILRSRAFSRLRNENLVVVPMADLINHSAGVTTEDHAYEVKGAAGLFSWDYLFSLKSPLSVKAGEQVYIQYDLNKSNAELALDYGFI

SEQ ID NO: 35 - protein, 255 aa, synthetic construct
  REGION 1..255: note = Synthetic or recombinant peptide
MLSPAVQTFWKWLQEEGVITAKTPVKASVVTEGLGLVALKDISRNDVILQVPKRLWINPDAVAASEIGRVCSELKPWLSVILFLIRERSREDSVWKHYFGILPQETDSTIYWSEEELQELQGSQLLKTTVSVKEYVKNECLKLEQEIILPNKRLFPDPVTLDDFFWAFGILRHRAESRLRNENLVVVPMADLEEHSAGVTTEDHAYEVKGAAGLFSWDYLFSLKSPLSVKAGEQVYIQGDLNKSNAELALDHGFI

SEQ ID NO: 36 - protein, 255 aa, synthetic construct
  REGION 1..255: note = Synthetic or recombinant peptide
MLSPAVQTFWKWLQEEGVITAKTPVKASVVTEGLGHVALKDISRNDVILQVPKRLWINPDAVAASEIGRVCSELKPWLSVILFLIREQSKEDSRWKHYFGILPWETDKTIYWSEEELQELQGSQLLKTTVSVKEYVKNECLKLEQEIILPNKRLFPDPVTLDDFFWAFGMCRHRAESRLRNENLVVVPMADLEEHSAGQTAEHHAYEVCGAAGQNSWDYLFSWTEPLNVKAGFQVYIQGDLIKSNAELYLDRGFI

SEQ ID NO: 37 - protein, 342 aa, synthetic construct
  REGION 1..342: note = Synthetic or recombinant peptide
MPFPYEFRELNPEEDKLVKANLGAFPTTYVKLGPKGYMVYRPYLKDAANIYNMPLRPTDVFVASYQRSGTTMTQELVWLIENDLNFEAAKTYMSLRYIYLDGFMIYDPEKQEEYNDILPNPENLDMERYLGLEYSSRPGSSLLAAVPPTEKRFVKTHLPLSLMPPNMLDTVKMVYLARDPRDVAVSSFHHARLLYLLNKQSNFKDFWEMFHRGLYTLTPYFEHVKEAWAKRHDPNMLFLFYEDYLKDLPGCIARIADFLGKKLSEEQIQRLCEHLNFEKFKNNGAVNMEDYREIGILADGEHFIRKGKAGCWRDYFDEEMTKQAEKWIKDNLKDTDLRYPNM

SEQ ID NO: 38 - protein, 342 aa, synthetic construct
  REGION 1..342: note = Synthetic or recombinant peptide
MPFPYEFRELNPEEDKLVKANLGAEPTTYVKLGPKGYMVYRPYLKDAANIYNMPLRPTDVFVASYQRSGTTMTQELVWLIENDLNFEAAKTYMSLRYIYLDGFMRYDPEKQEEYNDELPNPENLDMERYLGLEYSSRPGSSLLAAVPPTEKRFVKTHLPLSLMPPNMLDTVKMVYLARDPRDVAVSSFHHARLDYELNKQSNFKDFWEMFHRGLYTLTPYFEHVKEAWAKRHDPNMLFLFYEDYLKDLPGCIARIADFLGKKLSEEQIQRLCEHLNFEKFKNNGAVNMEDEREIGHEADGEHFIRKGKAGCWRDYFDEEMTKQAEKWIKDNLKDTDLRYPNM

SEQ ID NO: 39 - protein, 342 aa, synthetic construct
  REGION 1..342: note = Synthetic or recombinant peptide
MPFPYEFRELNPEEDKLVKANLDSEPDTEVKLGPKGYMVYFPDLKDAANIYNMPLRPTDVFVASYQRTGTTMTQELVWLIENDLNFEAAKTYMSLRYIYDDGFMRYFPEKLEAYNFELMIPRKPMSEKRYFLEGTVCIGSSLLAAVPPTEKRFVKTDLELSLMPPNMLDTVKMVYLARDPRDVAVSSFGNYRRDYSLCIQSNFKDFWEMFHRGLYTLVPYFEHVKEAWAKRHDPNMLFLFYEDYLKDLPGCIARIADFLGKKLSEEQIQRLCEHLNFEKFKNNGAVNMHDTRGQGVEADMIHRISKGKAGCWRDYFDEEMTKQAEKWIKDNLKDTDLRYPNM

SEQ ID NO: 40 - protein, 495 aa, synthetic construct
  REGION 1..495: note = Synthetic or recombinant peptide
MKIPKDTLIIAVENEIARINPAYSEDHDAVINLVFSGLTRFDENMSLKPDLAKSWDISKDGLVYDIFLRDDVLWHDGVKFSADDVKFSIEAFKNPKNNSSIYVNFEDIKSVEILNPSHVKITLFKPYPAFLDALSIGMLPKHLLENENLNTSSFNQNPIGTGPYKFVKWKKGEYVEFKANEHFYLDKVKTPRLIIKHIFDPSIASAELKNGKIDAALIDVSLLNIFKNDENFGILREKSADYRALMFNLDNEFLKDLKVRQALNYAVDKESIVKNLLHDYAFVANHPLERSWANSKNFKIYKYDPKKAEDLLVSAGFKKNKDGNFEKDGKILEFEIWAMSNDPLRVSLAGILQSEFRKIGVVSKVVAKPAGSFDYSKVDSFLIGWGSPLDPDFHTFRVFESSQDSALNDEGWNFGHYHDKKVDIALQKARNTSNLEERKKYYKDFIDALYENPPFIFLAYLDFALVYNKDLKGIKTRTLGHHGVGFTWNVYEWSK

SEQ ID NO: 41 - protein, 495 aa, synthetic construct
  REGION 1..495: note = Synthetic or recombinant peptide
MKIPKDTLIIAVENEIARINPAYSEDHDAVINLVFSGLTRFDENMSLKPDLAKSWDISKDGLVYDIFLRDDVLWHDGVKFSADDVKFSIEAFKNPKNNSSIYVNFEDIKSVEILNPSHVKITLFKPYPAFLDALSIGMLPKHLLENENLNTSSFNQNPIGTGPYKFVKWKKGEYVEFKANEHFYLDKVKTPRLIIKHIFDPSIASAELKNGKIDAALIDVSLLNIFKNDENFGILREKSADYRALMFNLDNEFLKDLKVRQALNYAVDKESIVKNLLHDYAFVANHPLERSWANSKNFKIYKYDPKKAEDLLVSAGFKKNKDGNFEKDGKILEFEIWAMSNDPLRVSLAGILQSEFRKIGVVSKVVAKPAGSFDYSKVDSFLEGHGSPLDPDFHTFRVFESSQDSALNDEGENRGHYHDKKVDIALQKARNTSNLEERKKYYKDFIDALYENPPFIFLAYLDFALVYNKDLKGIKTRTLGHHGVGFTWNVYEWSK

SEQ ID NO: 42 - protein, 495 aa, synthetic construct
  REGION 1..495: note = Synthetic or recombinant peptide
MKIPKDTLIIAVVNEPSRINPAYSWDHDCKANLVFSGLTRFDENMSLKPDLAKSWDISKDGLVYDIFLRDDVLWHDGVKFSADDVKFSIEAFKNPKNNSTQCKWAEDIKSVEILNPSHVKITLFKPYPAFLDALSIGMLPKHLLENENLNTSSFNQNPIGTGPYKFVKWKKGEYVEFKANEHFYLDKVKTPRLIICEMNQMFIASAELKNGKIDAALIDVSLLNIFKNDENFGILREKSADYRALMFNLDNEFLKDLKVRQALNYAVDKESIVKNLLHDYAFVANHPLERSWANSKNFKIYKYDPKKAEDLLVSAGFKKNKDGNFEKDGKILEFEIWQHSADPLRVSLAGILQSEFRKIGVVSKVVAKPRCVFDGKCVDSFLEGHGSPLDPDFHTFRVFESSQDSALNDDDANYGSDTDKKVDIALQKARNTSNLEERKKYYKDFIDALYENPPFIFLAYLDFALVYNKDLKGIKTRTLGHHGVGFTWNVYEWSK

SEQ ID NO: 43 - protein, 401 aa, synthetic construct
  REGION 1..401: note = Synthetic or recombinant peptide
MALKFNPLVSQPYKLASSARPPVSTFRSPKFLCLASSSSPALSSKEVESLKKPFTPPREVHLQVLHSMPPQKIEIFKSMEDWAEQNLLPHLKDVEKSWQPQDFLPDPASDGFEDQVKELRERARELPDDYFVVLVGDMITEEALPTYQTMLNTLDGVRDETGASPTSWAVWTRAWTAEENRHGDLLNKYLYLSGRVDMRQIEKTIQYLIGSGMDPRTENNPYLGFIYTSFQERATFVSHGNTARQAKEHGDLKLAQICGTIAADEKRHETAYTKIVEKLLEIDPDGTVVAFADMMRKKISMPAHLMYDGRDDNLFDNFSSVAQRLGVYTAKDYADILEFLVGRWRIESLTGLSGEGNKAQEYLCGLTPRIRRLDERAQARAKKGPKIPFSWIHDREVQLVD

SEQ ID NO: 44 - protein, 361 aa, synthetic construct
  REGION 1..361: note = Synthetic or recombinant peptide
MPAHLLQEEISSSYTTTTTITAPPSRVLQNGGGKLEKTPLYLEEDIRPEMRDDIYDPTYQDKEGPKPKLEYVWRNIILMSLLHLGALYGITLIPTCKIYTYIWVLFYYLMGALGITAGAHRLWSHRTYKARLPLRVFLIIGNTMAFQNDVFEWSRDHRAHHKFSETDADPHNSRRGFFFSHVGWLLVRKHPAVKEKGSTLNLSDLRAEKLVMFQRRYYKPGVLLLCFILPTLVPWYLWDETFQNSLFFATLFRYALGLNVTWLVNSAAHMYGYRPYDKTINPRENILVSLGAAGEGFHNYHHTFPYDYSASEYRWHINFTTFFIDCMAAIGLAYDRKKVSKAAILARIKRTGEESYKSGVD

SEQ ID NO: 45 - protein, 398 aa, synthetic construct
  REGION 1..398: note = Synthetic or recombinant peptide
MALKLNPFLSQTQKLPSFALPPMASTRSPKFYMASTLKSGSKEVENLKKPFMPPREVHVQVTHSMPPQKIEIFKSLDNWAEENILVHLKPVEKCWQPQDFLPDPASDGFDEQVRELRERAKEIPDDYFVVLVGDMITEEALPTYQTMLNTLDGVRDETGASPTSWAIWTRAWTAEENRHGDLLNKYLYLSGRVDMRQIEKTIQYLIGSGMDPRTENSPYLGFIYTSFQERATFISHGNTARQAKEHGDIKLAQICGTIAADEKRHETAYTKIVEKLFEIDPDGTVLAFADMMRKKISMPAHLMYDGRDDNLFDHFSAVAQRLGVYTAKDYADILEFLVGRWKVDKLTGLSAEGQKAQDYVCRLPPRIRRLEERAQGRAKEAPTMPFSWIFDRQVKLVD

SEQ ID NO: 46 - protein, 392 aa, synthetic construct
  REGION 1..392: note = Synthetic or recombinant peptide
MALKLCFPPHKMPSFPDARIRSHRVFMASTIHSPSMEVGKVKKPFTPPREVHVQVTHSLAPEKREIFNSLNNWAQENILVLLKDVDKCWQPSDFLPDSASEGFDEQVMELRKRCKEIPDDYFIVLVGDMITEEALPTYQTMLNTLDGVRDETGASLTPWAIWTRAWTAEENRHGDLLNKYLYLSGRVDMKQIEKTIQYLIGSGMDPRTENNPYLGFIYTSFQERATFISHGNTARLAKEHGDLKLAQICGIIAADEKRHETAYTKIVEKLFEIDPDGTVLALADMMRKKVSMPAHLMYDGQDDNLFENFSSVAQRLGVYTAKDYADILEFLVGRWDIEKLTGLSGEGRKAQDYVCTLPPRIRRLEERAQSRVKKASATPFSWIFGREINLVD

SEQ ID NO: 47 - protein, 400 aa, synthetic construct
  REGION 1..400: note = Synthetic or recombinant peptide
MSFVKDFKPQALGDTNLFKPIKIGNNELLHRAVIPPLTRMRALHPGNIPNRDWAVEYYTQRAQRPGTMIITEGAFISPQAGGYDNAPGVWSEEQMVEWTKIFNAIHEKKSFVWVQLWVLGWAAFPDNLARDGLRYDSASDNVFMDAEQEAKAKKANNPQHSLTKDEIKQYIKEYVQAAKNSIAAGADGVEIHSANGYLLNQFLDPHSNTRTDEYGGSIENRARFTLEVVDALVEAIGHEKVGLRLSPYGVFNSMSGGAETGIVAQYAYVAGELEKRAKAGKRLAFVHLVEPRVTNPFLTEGEGEYEGGSNDFVYSIWKGPVIRAGNFALHPEVVREEVKDKRTLIGYGRFFISNPDLVDRLEKGLPLNKYDRDTFYQMSAHGYIDYPTYEEALKLGWDKK

SEQ ID NO: 48 - protein, 395 aa, synthetic construct
  REGION 1..395: note = Synthetic or recombinant peptide
MASMVAFRPEAFLCFSPPKTTRSTRSPRISMASTVGPSTKVEIPKKPFMPPREVHVQVTHSMPPQKIEIFKSLEDWAENNILVHLKPVEKCWQPQDFLPDPSSEGFHEEVKELRERSKEIPDDYYVCLVGDMITEEALPTYQTMLNTLDGVRDETGASLTSWAVWTRAWTAEENRHGDLLNKYLYLSGRVDMKQIEKTIQYLIGSGMDPRTENSPYLGFIYTSFQERATFISHGNTARHAKEHGDVKLAQICGTIASDEKRHETAYTKIVEKLFEIDPDGTVLSFADMMKKKISMPAHLMYDGQDDNLFEHFSAVAQRLGVYTAKDYADILEFLINRWKVGELTGFSGEGKRAQDFVCTLAPRIRRIEERAQERAKQAPRIPFSWIYGREVQLVD

SEQ ID NO: 49 - protein, 495 aa, synthetic construct
  REGION 1..495: note = Synthetic or recombinant peptide
MEQPQAMEKVESVDVTEANAVAAGTNKLTKRIVAAGLGGKLMGTKSMVDVTADQLAKDSINELLARDKALKEKYEKQKHISEQPWTKDNWHQHINWLNMILVCGLPLFGAIATYYKPPTKETVILGVVLYILGGLSITAGYHRLWSHRAYSARTPLKVLFALFGAGAVEGSIKWWSHSHRVHHRYTDTHRDPYDARKGFWYSHMGWMLTKPNPRYKARADISDLADDWIVRFQHRHYLLIMTFMALILPTIIAKYFWDDAWGGFIYGGIFKVFVIQQATFCVNSLAHWIGVQPFDDRRTPRDHFLTAIVTFGEGYHNFHHEFPSDYRNALKWYQYDPTKIVIYLSSKVGLSYNLKTFSDNAIKQGLVQQQQKKLDSMRAHLNWGTPLQDLPVWDKSEFFEKSKDKKGLVIISGIVHNVENFIGEHPGGEKLVKGALGKDATTAFNGGVYAHSNAAHNLLATMRVAVIRDGNANANTFSLQNEFLDKKAAAAAASN

SEQ ID NO: 50 - protein, 494 aa, synthetic construct
  REGION 1..494: note = Synthetic or recombinant peptide
MDIPTAKYSEEDMKAALQPTEPEFFKVKVSKKKKNRRKVKHDTTASTIRRHISESKWTISNWYRHIHWKNVVLVVIVPLLALLGSLTVPLTRTGFYWMGFNYIITCTFVIMGYHRYWAHRSFQTTKEMSFLFAMVGASAGVGSAKWWCASHRAHHRFCDTERDPHNIRKGFWYSHFGWMLLIHHPKVQAAIKESESEDISSDPVILWQYENYFQMFLVTGILIPAIIGWWLFQDFAGGLVYGGLVKIFLVQNTIFSVNSLCHCCGSQPFDDAKSSRNSILLSLITFGEGQHNFHHEFPSDCRNGVEWYDFDPVKWVIFTLNALGVIYNVHAAPTTAINQLRVQQAQRILDKERSQLNWGIRIDKLPTLTSAEFSKLAKESQPQRALVVISGIVHDVTPFLHDHPGGVALIKSSIGKDATNAFNGAVYSHSNAARNLLATMRIAVILGSEQEVWKQQQKENKDVPLKNDSEGKKIVRSGEQITMTKTPSTTAGAA

SEQ ID NO: 51 - protein, 420 aa, synthetic construct
  REGION 1..420: note = Synthetic or recombinant peptide
MSAVTVTGNNGDASRSNTTTTTKRTGNVSSFSQSKGLTAIDTWGNVFKVPDFTIKQILDAIPKHCYERRLTTSFYYVFRDIFLIGCTMFMGSFIPMIENVFLRGAAYAALVFLLSVEYTGLWVLAHECGHQAFSDYGWVNDTVGWILHSYLLVPYFSWKYSHGKHHKATGHLTRDMVFVPATKEKFLEKRNASKLGELGEDAPIFTLYQLVAQQLGGWILYLFTNVTGQPYPNTPKWMQNHFVPSSPIFEKKDYWFIILSDLGILAQLMVLYVWRQQMGNWNLFIYWFLPYVLTNHWLVFITFLQHSDPTMPHYEAEQWTFARGAAATIDREFGFIGPFFFHDIIETHVLHHYVSRIPFYNAREASEGIKKVMGEHYRYSGENMWVSLWKSGRSCQFVDGENGVKMYRNINNWGIGTGEK

SEQ ID NO: 52 - protein, 415 aa, synthetic construct
  REGION 1..415: note = Synthetic or recombinant peptide
MSKVTVSGSEILEGSTKTVRRSGNVASFKQQKTAIDTFGNVFKVPDYTIKDILDAIPKHCYERSLVKSMSYVVRDIVAISAIAYVGLTYIPLLPNEFLRFAAWSAYVFSISCFGFGIWILGHECGHSAFSNYGWVNDTVGWVLHSLVMVPYFSWKFSHAKHHKATGHMTRDMVFVPYTAEEFKEKHQVTSLHDIAEETPIYSVFALLFQQLGGLSLYLATNATGQPYPGVSKFFRSHYWPSSPVFDKKDYWYIVLSDLGILATLTSVYTAYKVFGFWPTFITWFCPWILVNHWLVFVTFLQHTDSSMPHYDAQEWTFAKGAAATIDREFGILGIIFHDIIETHVLHHYVSRIPFYHAREATECIKKVMGEHYRHTDENMWVSLWKTWRSCQFVENHDGVYMFRNCNNVGVKPKDT

SEQ ID NO: 53 - skipped